(19) United States
(12) Patent Application Publication
(10) Pub. No.: US 2002/0132370 A1
(43) Pub. Date: Sep. 19, 2002

(54) Detection of a blood coagulation activity marker in a body fluid sample

(76) Inventors: Michael Rud Lassen, Rungsted Kyst (DK); Lars C. Borris, Arhus C (DK)

Correspondence Address: BROWDY AND NEIMARK, P.L.L.C., 624 Ninth Street, N.W., Washington, DC, 20001, US

(21) Appl. No.: 09/791,384

(22) Filed: Feb. 23, 2001

Related U.S. Application Data
(60) Provisional application No. 60/211,390, filed on Jun. 14, 2000.

(30) Foreign Application Priority Data
Feb. 23, 2000 (DK) PA 2000 00283

(51) Int. Cl. G01N 33/53; G01N 33/543; C12Q 1/56

(52) U.S. Cl. 436/512; 436/518; 436/69; 435/7.9; 435/13; 422/56

(57) ABSTRACT

The invention relates to a method for detecting in a body fluid sample at least one blood coagulation activity marker that reflects e blood coagulation activity of an individual. By correlating the amount or concentration of the blood coagulation activity marker present e.g. in a urine sample, it is possible to monitor the blood coagulation activity of a patient following surgery without having to obtain a blood sample from said patient.

### TECHNICAL FIELD

The invention relates to a method for determining or monitoring the blood coagulation activity of an individual. More particularly, the invention pertains to a method for detecting in a body fluid sample at least one blood coagulation activity marker capable of indicating the blood coagulation activity of an individual. By correlating the amount or concentration of the blood coagulation activity marker present e.g. in a urine sample to the blood coagulation activity of an individual, it is possible e.g. to monitor the blood coagulation activity of a patient following surgery without having to obtain and analyse a blood sample from said patient.

### BACKGROUND OF THE INVENTION

The ability of an individual to selectively form blood clots in areas of trauma is of vital importance. Failure of the blood to clot may lead to severe haemorrhage and in some instances the lack of blood clotting may be fatal. However, an uncontrolled clotting or coagulation of the blood Within vessels can also lead to serious complications such as thrombosis.

Formation of a blood clot is a complicated process involving a large number of blood components designated clotting factors and platelets, which culminate in the formation of a fibrin clot. Cascades of reactions eventually results in convertion of prothrombin in the blood to As enzymically active form thrombin. Thrombin catalyses the formation of the insoluble protein fibrin from soluble fibrinogen; the fibrin forms a fibrous network in which blood cells become enmeshed, producing a clot.

It has long been recognised that many clinical conditions result in improper levels of for instance fibrinogen, prothrombin or thrombin in the blood. The improper levels may in turn lead to the development of hypo- or hypercoagulant states of bleeding and clotting. For instance, states of hypo-fibrinogenemia or hyper-fibrinogenemia may result from hepatic disease, from disseminated intravascular coagulation, from fibrinolytic syndrome, neoplastic disease, and post-operatively due to trauma.

Since thrombin catalyses the formation of fibrin from fibrinogen, thrombin activity is directly responsible for the coagulation of blood or plasma, and the conversion of pro-thrombin to thrombin is thus a key event in the coagulation of blood. One aspect of the present invention is concerned with monitoring this key event. The intrinsic pathway of blood clot formation involves coagulation factors, that circulate in the form of inactive precursors. Upon activation they are converted into an active form, which in turn activates the next clotting factor in sequence. In this way, the inactive proenzyme Factor XII is converted to the active enzyme XIIa which in turn converts the zymogen Factor XI to the enzyme Factor XIa, which then activates Factor IX in the presence of calcium. The enzyme Factor IXa activates Factor X in the presence of Factor VIII and phospholipid. This reaction is greatly increased by the prior exposure of Factor VIII to thrombin or Factor Xa.

In the extrinsic pathway, Factor X can be activated by either a complex of thromboplastin and Factor VII, or a complex of platelet phospholipid activated Factor IX and Factor VIII. Activated Factor X, in the presence of calcium, Factor V and platelet phospholipid, activates Factor II (pro-thrombin) which is cleaved to form thrombin which converts Factor I (fibrinogen) to fibrin in blood plasma.

The process of blood coagulation is modified by a number of positive and negative feed back loops and by interaction between the pathways. For example, thrombin and Factor Xa, formed either by activation of the intrinsic or extrinsic pathway, feed back to activate Factor VIII and Factor V. Factor Xa feeds back to initially increase and then to inhibit its own activation by Factor VIIa. The intrinsic and extrinsic pathways are also linked For example, Factor VII is activated by Factor IXa, XIIa and XIa and Factor VIIa can activate Factor IX.

Activation of clotting leading to the conversion of the proenzyme pro-thrombin into the active protease thrombin is of particular interest for the present invention. Thrombin itself increases the rate of its production by activating the cofactors factor V and factor VII proteolytic cleavage. These activated cofactors form, with the proteases factor Xa and Ixa, active enzyme/cofactor complexes on phospholipid surfaces, the activity thereof being a factor of about 1000 higher than that of the proteases alone. This positive feedback results in almost explosive production of large amounts of thrombin. Thrombin converts fibrinogen into fibrin which, in the normal case, leads to wound closure and wound healing. In order to prevent life-threatening spreading of the clotting, which would lead to blockage of the vascular system in the body, i.e. thrombosis, it is necessary to inhibit both the active protease and the resupply of protease. Active proteases are neutralized in the body by protease inhibitors by the formation of covalent complexes. The stoppage of replenishment is initiated by thrombin itself. For this purpose, thrombin binds to the membrane protein thrombomodulin and converts the proenzyme protein C into the active protease protein Ca (APC). APC in turn forms, with the cofactor protein S (PS), a complex which proteolytically cleaves, and thus inactivates, the active cofactors factor VIIIa and Va. APC thereby eliminates the stimulation exerted by these cofactors.

The level of thrombin present in vivo is primarily regulated by the heparin-catalysed thrombin inhibitor, antithrombin III (ATIII). Hence, the level of ATIII present in vivo is also of significant clinical importance for diagnosing and monitoring patients at risk for excessive bleeding, due to abnormally high levels of ATIII, or at risk for developing thrombi, due to abnormally low levels of ATIII. Although blood and plasma contain ATIII, ATIII alone is a relatively weak inhibitor of thrombin. However, ATIII is activated when being bound to heparin, and the activated ATIII is a potent inhibitor of the proteolytic activity of thrombin. Consequently, heparin is often administered to patients with risk of thrombosis. A precise adjustment of the heparin concentration is extremely important. If the dose of heparin is too low there is the danger of thrombosis or embolism, and if the dose is too high, excessive bleeding may result,

Substantial efforts have been made to measure clotting components or evaluating the blood coagulation activity. Most methodologies rely upon immunologic and clotting techniques although clearly the latter is preferred. The immunologic techniques, although generally capable of precisely defining the levels of the various components within the blood stream, are generally incapable of distinguishing between active and inactive forms of blood coagulation factors. Accordingly, the immunologic methods are often described as being less accurate with respect to the patient's actual clotting ability. Consequently, the results obtained by clotting techniques are often preferred among medical staff and perceived as being clinically more significant.

The basis of in vitro testing of blood coagulation has commonly been a determination of changes in turbidity, viscosity or electrical conductivity of a blood sample caused by the conversion of fibrinogen to fibrin during clot formation. Accordingly, a normal blood sample tend to produce a strong gel clot, whereas samples producing thin, watery, webby-type clots are indicative of some coagulation abnormality. The screening tests for coagulation disorders routinely include the pro thrombin time (PT) and the activated partial thromboplastin time (APTT) Automated coagulation instrumentation, both mechanical and optical density-based, provide data about the end point of the clotting times in the various coagulation tests. The fibrometer-type of instrument measures increasing conductivity which may be correlated to the formation of clots. Essentially, the screening tests for coagulation disorders are designed to detect a significant abnormality in one or more of the clotting factors and to localise this abnormality to various steps in the coagulation pathway.

APTT measures coagulation factors of the intrinsic pathway, including Factors XII, XI, IX, VIII, X, V, II and I which may be abnormal due to heritable disorders or heparin therapy. APTT is therefore useful as a presurgical screen and for monitoring heparin therapy. The APTT is typically performed by adding an activator such as kaolin, ellagic acid, or silica, for example, with phospholipid to a plasma sample. This activates Factors XII and XI. Phospholipid substitutes for platelet in the activation of Factor VIII by Factors IX, VIII and V. Blood coagulation is initiated in this clotting test by adding calcium. Factor VII is the only factor not affected by the partial thromboplastin time and the APTT is, therefore, normal in patients with a Factor VII deficiency.

The pro-thrombin time (P) test is performed by adding tissue thromboplastin with calcium to plasma. This initiates clotting by activating Factor VII which in turn activates Factor X which in the presence of Factor V, converts pro-thrombin to thrombin and the thrombin which is so produced converts fibrinogen to fibrin. PT therefore bypasses the intrinsic clotting pathway and is normal in patients with deficiencies of Factors XII, XI, IX and VIII. PT is abnormal in patients with deficiencies of Factors VII, X, V, pro-thrombin or fibrinogen.

The normal PT or APTT tests have found widespread acceptance despite the fact that each test has associated therewith a level of indefiniteness regarding the point at which the clot is determined to have occurred.

Another generally known coagulation test procedure is the Activated Whole Blood Coagulation Time (AWBCT). Typical known AWBCT tests are performed by placing a whole blood specimen in a test tube containing solid particulate material such as celite for activation of Hagemann Factor. Thereafter, the sample is heated and agitated, and the time necessary for the sample to clot is measured. As with the activated partial thromboplastin time (APTT) tests described herein above, the AWBCT tests often give unreliable and unreproducible results.

More advanced instruments, such as the KoaguLab.RTM. (Ortho Diagnostic Systems Inc., Raritan, N.J.) generates a printed graph of the clotting reaction. Clinicians can tell by the shape of the curve generated whether or not the clotting times is reliable, thus providing a stronger information bass for their therapeutic decisions. A graph which plots turbidity against reaction time is referred to as “clot signature”. KoaguLab.RTM. may be used to perform PT and APTT assays. These are performed by adding brain thromboplastin or activated partial thromboplastin and calcium chloride respectively, to a plasma sample and determining the time at which the clot forms. The clot signature essentially adds a qualitative fibrinogen measurement to the standard PT and APTT tests, which may prove useful in detecting certain disease states, including hypercoagulability.

The following prior art documents describes various methods for measuring blood coagulation activity or blood coagulation markers:

U.S. Pat. No. 5,169,786 relates to a method for determining both extrinsic and intrinsic clotting factors as well as protein C in blood. The method is based on factor-based assays exploiting either the prothrombin time test (PT) or the activated partial thromboplastin time test (APTT), and the observed rate of clot formation (Velocity) and the first derivative of the observed rate of clot formation (Acceleration) are determined in test samples and compared with normal plasma samples. The Velocity or Acceleration value in the test sample can be compared directly with the Velocity or Acceleration value in a normal plasma sample, Also, an individual factor level can be correlated with that factor's Velocity and Acceleration in a test sample, which is compared with known, normal ranges. A pro-thrombin time test (PT) is used to determine deficiencies of clotting factor activity in the extrinsic pathway. An activated partial thromboplastin time test (APTT) is used to indicate abnormalities in most of the procoagulant clotting factors. The APTT assay is a useful sensitive procedure for generating heparin response curves and for screening deficiencies of clotting factors in the intrinsic pathway.

U.S. Pat. No. 5,443,960 relates to a method for screening and diagnosis of thromboembolic diseases based on a determination of activated protein C (APC) resistance detected i) by a low anti-coagulant response to exogenous APC that is not related to a Protein S deficiency or deficient FVIII/FVIIIa, and ii) by a low anti-coagulant response to exogenous APC in the absence of APC immunoglobulin inhibitors. The disclosed method comprises the steps of i) incubating a human plasma sample with exogenous APC, or exogenous Protein C and an exogenous reagent transforming exogenous Protein C to APC, and an exogenous reagent at least partially activating a coagulation factor of the blood coagulation system of said human plasma sample, ii) measuring a substrate conversion rate for a coagulation factor directly or indirectly activated in step i), and iii) comparing said substrate conversion rate measured in step ii) with a standard value obtained from samples of normal individuals having been subjected to steps i) and ii).

U.S. Pat. No. 5,726,028 describes the detection of disturbances of the protein C/protein S system in blood by means of a functional clotting test wherein endogenous protein C in the sample is activated by adding a protein C activator to the sample. This normally leads to prolongation of the clotting time, presumably because of the breakdown of the activated cofactors factor Va and factor VIIIa. A less pronounced prolongation of the clotting time indicates a disturbance of the system, for which reason the test is also described as being suitable as a screening test.

U.S. Pat. No. 5,716,795 describes a one-stage assay using soluble thrombomodulin for directly determining the functional status of the protein C system in plasma. The activity of the protein C system is used to determine the risk of thrombosis in the host individual. In another embodiment the assay reveals the existence of an additional component in protein C activation, and thus the existence of an additional component in the regulation of blood coagulation.

U.S. Pat. No. 5,292,664 describes a method for determining fibrinogen from undiluted plasma samples. The use of undiluted plasma as sample is made possible by the use of a specific peptide inhibitor of fibrin aggregation in a concentration which permits aggregation of fibrin but with reduced speed, so that coagulation time can be measured easily. Undiluted plasma, is incubated with a reagent containing i) at least one inhibitor of fibrin aggregation in an amount effective to increase the coagulation time to allow measurement of the fibrin concentration, and ii) thrombin, or a protease having a similar activity, in an amount, which immediately converts the fibrinogen into soluble fibrin. The method thus makes it possible to determine the coagulation time.

U.S. Pat. No. 5,985,582 relates to an evaluation of the hemostasis of a patient by determining the level of antithrombin III (ATIII) present in a plasma sample withdrawn from a patient. The thrombin-based assay for determining ATIII present in a plasma sample involve using a heparin derivative effectively enhancing the antithrombin activity of ATIII. The assay comprises the steps of i) combining the plasma sample with thrombin and with a heparin derivative to form an assay mixture, ii) forming a complex between the ATIII and the thrombin in the assay mixture, iii) determining the uncomplexed thrombin in the assay mixture, and iv) correlating the determined uncomplexed thrombin with ATIII in the plasma sample.

U.S. Pat. No. 5,648,228 is related to a method for measuring the activity of tested substances utilizing a reconstituted plasma kallikrein-kinin system. A series of enzymatic reactions is started wherein an activation of a blood coagulation factor XII is an initiating reaction. The series of reactions is started in the presence of the tested substance in the reconstituted plasma kallikrein-kinin system Then, the series of reactions is stopped and the physiologically active substance produced in the reaction series is quantitatively determined. The method of measuring the activity is useful for adjusting the plasma kallikrein-kinin system, the blood clotting system, and the fibrnolysis system.

U.S. Pat. No. 4,463,090 describes an enzyme immunoassay wherein the sensitivity is increased by means of a cascade amplification. The coupled ligand in the form of an enzyme or an activator catalytically activates a second enzyme that may act on a substrate or on a third enzyme to produce the cascade. Alternatively, a proenzyme is coupled to the ligand and converted by an activator to an enzyme which is itself an activator of a second proenzyme in the cascade reaction. Markers such as fibrin and kinin are measured by means of using suitable proenzymes, enzymes and activators.

Slaughter et al. (1994) Anesthesiology 80(3), 520-526, measured pro-thrombin activation during the perioperative period in 19 adults undergoing primary cardiac surgery. Enzyme-linked immunosurbent assays were used for the detection of thrombin formation (pro-thrombin fragment 1+2 and thrombin-antithrombin III complex) and thrombin activity (fibrinopeptide A and fibrin monomer). Blood samples were obtained preoperatively, during cardiopulmonary bypass surgery, and in the postoperative period. It was observed that despite administration of heparin, plasma concentrations of prothrombin fragment 1+2, thrombin-antithrombin III complex, and fibrin monomer increased throughout surgery. Peak concentrations for all hemostatic markers occurred in samples obtained 3 hours postoperation. Markers for thrombin activity, however, suggested the presence of active thrombin through the morning after surgery. It was suggested that further analysis would be necessary in order to determine the role of hemostatic activation in thrombotic complications after cardiac surgery.

Further prior art methods for analysing a blood or plasma sample in order to detect the blood coagulation activity of a patient is described by Corradi et al. (1999) in Acta Orthop. Beig. 65(1), p. 39-43 (Preoperative plasma levels of pro-thrombin fragment 1+2 correlate with the risk of venous thrombosis after elective hip replacement), by Li et al. (1999) in J. Am. Coll. Cardiol. 33(6), p. 1543-1548 (Prognostic significance of elevated hemostatic markers in patients with acute myocardial infarction), by Brack et al. (1993) in Int. J. Cardiol. 38(1), p. 57-61 (Pro-thrombin fragment F1+2 concentrations for monitoring anticoagulation therapy with heparin), by Bruhn and Zurborn (1995) in J. Heart Valve Dis. 4(2), p, 138-140 (The use of pro-thrombin fragment F1+2 to monitor the effect of oral anticoagulation), by Suzuki et al. (1993) in Rinsho Byori, 41(2), p. 215-219 (Evaluation of an enzyme-linked immunosorbent assay for the determination of pro-thrombin fragment F1.2 (Dade Pro-thrombin Fragment F1.2 ELISA: Baxter Diagnostics Inc., U.S.A. using micro-titer plate [Article in Japanese]), by Butenas et al. (1999) in Blood 94(7), p, 2169-2178 (“Normal” thrombin generation), and by Giannitsis et al. (1999) in Int. J. Cardiol. 68(3), p. 269-274 (Pro-thrombin fragments F1+2, thrombin-antithrombin III complexes, fibrin monomers and fibrinogen in patients with coronary atherosclerosis).

Apart from the above-mentioned methods and assays based on analysis of blood or plasma samples, the prior art also contains references to the detection of blood coagulation markers in samples of body fluids such as urine.

U.S. Pat. No. 3,853,710 and U.S. Pat. No. 3,960,669 relate to a method of detecting an abnormal concentration of fibrinolytic enzymes and fibrinogen degradation products in the blood of an individual, such an abnormal concentration being characteristic of certain pathologic states, comprising the steps of determining the average and the range of clotting times of standardized saline solutions of buffered thrombin, fibrinogen and urine from healthy subjects combined in selected proportions at a selected temperature, and determining the individual clotting time at the same selected temperature of the same standardized saline solutions of buffered thrombin and fibrinogen with a urine specimen from said individual combined in said selected proportions, an individual clotting time deviating by a selected amount from said average being taken as indicative of an abnormal concentration of fibrinolytic enzymes and fibrinogen degradation products in the blood and certain characteristic pathologic states. Accordingly, the method is capable of determining a variation from the normal concentration of fibrinolytic enzymes and FDP in the blood of an individual. There is also disclosed a method for determining the presence in an individual of a malady of such a type as causes a change in fibrinolytic enzymes and FDP in the blood, such maladies including cancer, hepatitis, liver malfunction and blood clots, coronary thrombosis, cerebral thrombosis, deep vein thrombosis and pre-infarction syndrome.

Sorensen et al. (1992) Thrombosis Research 67, 429-434, discloses the detection of pro-thrombin fragments 1 and 2 and fibrinopeptide A in urine samples obtained from healthy individuals and from individuals with multiple trauma. The obtained data were not conclusive, and it is suggested that further studies should be carried out in order to validate the performed measurements and to evaluate the possible clinical use of the seemingly sensitive test for coagulation activation.

Bezeaud and Guillin (1984) British Journal of Haematology 58(4), 597-506, discloses radioimmunoassays for the detection of pro-thrombin fragments 1 and 2 in urine samples. It is stated that the significant increase in fragment 1 and fragment 2 excretion observed in a condition known to be associated with the hypercoagulable state suggests that the measurement of pro-thrombin derivatives in urine could be a useful tool for the non-invasive detection of thromboembolic diseases or prethrombotic states.

Lind et al, (1999) Blood Coagulation Fibrinolysis 10(5), 285-289, investigated the possibility of using randomly collected urine samples as non-invasive means of assessing the state of coagulation system activation. Using a commercially available enzyme-linked immunosorbent assay kit designed to measure plasma levels of pro-thrombin fragment 1+2, they reported the detection of immunoreative pro-thrombin fragment 2 in healthy individuals, and significantly increased levels in diabetic and non-diabetic pregnant women, and individuals with venous thromboembolism, prostate cancer, and diabetes. It is suggested that measurements of excretion of immunoreactive fragment 2 are worth a further study as an adjunct or alternative to plasma-based assays designed to detect or quantify coagulation system activation.

Tripodi et al. Tthromb. Haemost. evaluated the pattern of pro-thrombin fragment 1+2 changes as a function of increased intensity of anticoagulation, The studies confirmed previously obtained results, and it was concluded that the results indicated that measurement of pro-thrombin fragment 1+2 is not a suitable laboratory tool to monitor oral anticoagulants.

Further studies are reproted by Leeksma et al. (1985) in Thromb. Haemost. 54(4), p. 792-798 (Fibrinopeptide A in urine from patients with venous thromboembolism. disseminated intravascular coagulation and rheumatoid arthritis—evidence for desphorylation and carboxyterminal degradation of peptide by the kidney), and by Gallino et al. (1985) in Thromb. Res. 39(2), p. 237-244 (Fibrinopeptide A excretion in urine in patients with atherosclerotic artery disease).

Nowhere does the prior art disclose a non-invasive method for determining or monitoring the blood coagulation activity of an individual. Also, the prior art does neither teach a method for detecting in a body fluid sample at least one blood coagulation activity marker capable of indicating the blood coagulation activity of an individual, nor does it disclose a method of correlating the amount or concentration of a blood coagulation activity marker present e.g. in a urine sample with the blood coagulation activity of a patient. In contrast, the prior art concerned with determining the blood coagulation activity of an individual is exploiting time-consuming and expensive assays for analysing blood or plasma samples.

### SUMMARY OF THE INVENTION

There exists a need for more sensitive, accurate and reliable blood coagulation activity assays that can be used to determine coagulative properties of blood and plasma In particular, there is a need for economical, non-invasive assays for accurately determining or monitoring clotting conditions for which there currently exists neither accurate nor reliable tests. There is also a need for even more sensitive blood clotting tests which give consistent and reproducible results.

Following invasive therapy, such as ordinary surgery, the blood coagulation activity of a patient is likely to result in an increased risk of e.g. thrombosis, and anti-coagulants are often prescribed by the medical staff. The anticoagulants are prescribed to protect patients from e.g. the formation or presence of a clot in a blood vessel. Accordingly, in one aspect the present invention is directed to a determination of the blood coagulation activity of an individual, said determination being used for monitoring the risk of thrombosis in individuals having undergone surgery. The present invention in another aspect is concerned with a method for monitoring a thromboembolic disease.

Anti-coagulants are often prescribed indiscriminately in the post-operative phase irrespective of whether the patient is in need of anti-coagulant treatment due to the lack of accurate and reliable practical monitoring methods. The indiscriminate use of anti-coagulants is expensive and a need exists for monitoring e.g. heparin, acetylsalicylic acid (aspirin), and various coumarin derivatives. The invention solves this problem by identifying the patients who are in need of anti-coagulant treatment in the post-operative phase.

The present invention provides a method and a system capable of providing reliable information about blood coagulation activity by assessing a blood coagulation activity marker in a body fluid sample. This means that there is a significant correlation between the blood coagulation activity and the concentration of the blood coagulation activity marker in the body fluid sample.

The present invention relates to a method for detecting in a body fluid sample, such as a urine sample, at least one blood coagulation activity marker that is correlatable with the blood coagulation activity of an individual. By correlating the amount or concentration of the blood coagulation activity marker present in the sample with the blood coagulation activity of an individual, it is possible to monitor said blood coagulation activity of said individual.

The invention also relates to a kit for detection of said blood coagulation activity marker in e.g. a urine sample. The kit can be used as part of a home patient management programme. In one aspect the programme provides a means for monitoring the blood coagulation activity of an individual in a post surgery phase.

Being able to monitor the blood coagulation activity as part of a home patient management programme means that the individual does not have to consult a medically trained expert, who would otherwise be needed to obtain and analyse a blood sample before an assessment of the blood coagulation activity could be made. The conventional analysis of a blood sample and the assessment of the blood coagulation activity of an individual is both expensive and time consuming. Also, if it in fact turns out that no need exists for adjusting the blood coagulation activity of an individual, the expensive and time consuming blood sample analysis and assessment of the blood coagulation activity may actually have been done in vain. In contrast, the present invention makes it possible to initially screen—by means of the non-invasive assay method of the present invention—a large number of individuals potentially in need of having their blood coagulation activity regulated by e.g. administration of a medicament having an anticoagulating effect. In contrast, a conventional treatment may involve administration of an anticoagulation medicament to each and all of said individuals since no possibility exists for readily determining the specific individuals who are actually in need of administration of an anticoagulation medicament. This is particularly the case following surgery, where an anticoagulation medicament such as heparin is very often administered indiscriminately to each and every patient having undergone surgery,

The invention also facilitates a more efficient use of health care ressources by reducing the period post surgery during which a patient will have to remain hospitalised in order for the hospital staff to monitor the blood coagulation activity of the patient The invention makes it possible for patients having undergone surgery to be discharged from the hospital and having their blood coagulation activity monitored in their own home as part of a home patient management programme. Monitoring the blood coagulation activity of an individual during a post operational phase makes it possible to reduce the period of time during which a patient is admitted to hospital. The home patient management programme may further involve the transmission of data and results recorded by the patient to a hospital unit where the data and results can be monitored more carefully by medically trained personel. In this way it is possible to keep track of all home patients and optionally only admit to a hospital the patients in need of treatment.

In a first aspect the present invention relates to a method for correlating a predetermined amount of at least one blood coagulation activity marker comprised in a sample with the amount of at least one quantifiably detectable reporter species capable of being operably linked to said blood coagulation activity marker, said method comprising the steps of

i) obtaining a test sample comprising a predetermined amount of at least one blood coagulation activity marker,

ii) obtaining at least one quantifiably detectable reporter species capable of being operably linked to said blood coagulation activity marker,

iii) contacting said test sample comprising said predetermined amount of at least one blood coagulation activity marker with said at least one quantifiably detectable reporter species,

iv) operably linking said predetermined amount of said blood coagulation activity marker comprised in said test sample to said at least one quantifiably detectable reporter species,

v) detecting said at least one quantifiably detectable reporter species operably linked to said predetermined amount of said blood coagulation activity marker comprised in said test sample,

vi) determining the amount of said at least one quantifiably detectable reporter species operably linked to said predetermined amount of said blood coagulation activity marker comprised in said test sample, and

vii) correlating said predetermined amount of said blood coagulation activity marker comprised in said test sample with said determined amount of said at least one quantifiably detectable reporter species.

By following the steps outlined above it is possible to produce an assay for determining a blood coagulation activity marker by detecting the amount of reporter species in the assay. By repeating the steps using different predetermined amounts of the at least one blood coagulation activity marker it is possible to produce an assay having more than one cut-off value, capable of determining various levels of coagulation activity,

Standard values obtained for example as described by the above method allow that for any given amount or concentration of reporter species detected it is possible to correlate to the amount of coagulation activity marker.

Thus, in another aspect of the present invention there is provided a method for determining the amount of at least one blood coagulation activity marker comprised in a body fluid sample, said method comprising the steps of

i) obtaining a body fluid sample comprising at least one blood coagulation activity marker,

ii) contacting said body fluid sample comprising said blood coagulation activity marker with at least one quantifiably detectable reporter species,

iii) operably linking said blood coagulation activity marker comprised in said body fluid sample to said at least one quantifiably detectable reporter species,

iv) detecting said at least one quantifiably detectable reporter species operably linked to said blood coagulation activity marker comprised in said body fluid sample,

v) determining the amount of said at least one quantifiably detectable reporter species operably linked to said blood coagulation activity marker comprised in said body fluid sample,

vi) correlating the determined amount of said at least one quantifiably detectable reporter species with the amount of said blood coagulation activity marker comprised in said body fluid sample, and

vii) based on the correlation of step vi), determining said amount of said blood coagulation activity marker comprised in said body fluid sample,

In a further aspect the invention relates to a method for correlating the blood coagulation activity of a blood sample obtained from an individual with the amount of at least one blood coagulation activity marker comprised in a body fluid sample obtained from said individual, said method comprising the steps of

i) obtaining a blood sample from said individual,

ii) obtaining a body fluid sample comprising at least one blood coagulation activity marker from said individual,

iii) determining the amount of at least one blood coagulation activity marker present in said body fluid sample obtained from said individual, and

iv) correlating said amount of said at least one blood coagulation activity marker present in said body fluid sample obtained from said individual with said blood coagulation activity of said individual.

In yet another aspect there is provided a method for correlating the blood coagulation activity of a blood sample obtained from an individual with the amount of at least one blood coagulation activity marker comprised in a body fluid sample obtained from said individual, said method comprising the steps of

i) obtaining a blood sample from said individual,

ii) obtaining a body fluid sample comprising at least one blood coagulation activity marker from said individual,

iii) determining the amount of at least one quantifiably detectable biological species present in said blood sample obtained from said individual, said at least one quantifiably detectable biological species being correlatable to said blood coagulation activity in said blood sample obtained from said individual,

iv) determining the amount of at least one blood coagulation activity marker present in said body fluid sample obtained from said individual, said blood coagulation activity marker being correlatable with said at least one quantifiably detectable biological species present in said blood sample obtained from said individual,

v) correlating said amount of said at least one blood coagulation activity marker present in said body fluid sample obtained from said individual with the amount of at least one quantifiably detectable biological species present in said blood sample obtained from said individual,

vi) correlating said amount of said at least quantifiably detectable biological species present in said blood sample obtained from said individual with the blood coagulation activity of said individual, and

vii) based on the correlations of steps v) and vi), correlating said amount of at least one blood coagulation activity marker present in said body fluid sample obtained from said individual with said blood coagulation activity of said individual.

In a further aspect the invention relates to a method for determining the blood coagulation activity of an individual, said method comprising the steps of

i) obtaining a body fluid sample comprising at least one blood coagulation activity marker from said individual,

ii) determining the amount of said at least one blood coagulation activity marker present in said body fluid sample,

iii) correlating said determined amount of said at least one blood coagulation activity marker present in said body fluid sample with said blood coagulation activity of said individual, and

iv) based on the correlation of step iii), determining said blood coagulation activity of said individual.

In a still further aspect there is provided a method for monitoring the blood coagulation activity of an individual, said method comprising obtaining a plurality of individual determinations of said blood coagulation activity of said individual, wherein each determination of said blood coagulation activity is obtainable by the method for determining said activity according to the invention

In yet another aspect the invention relates to a method for monitoring a clinical condition in an individual, said clinical condition affecting the blood coagulation activity in said individual, said method comprising the steps of

i) obtaining over a predetermined period of time a plurality of body fluid samples comprising at least one blood coagulation activity marker from said individual,

ii) determining the amounts of said at least one blood coagulation activity marker present in said plurality of body fluid samples,

iii) correlating said determined amounts of said at least one blood coagulation activity marker present in said plurality of body fluid samples obtained over a predetermined period of time with said clinical condition affecting said blood coagulation activity in said individual, and

iv) based on said correlation of step iii), monitoring said clinical condition in said individual.

The invention in yet another aspect provides a method for treating a clinical condition in a human or animal body by therapy or surgery, said method comprising the steps of

i) determining the amount of at least one blood coagulation activity marker comprised in a body fluid sample according to a method of the present invention,

ii) correlating said amount of said blood coagulation activity marker in said body fluid sample determined in step i) to said clinical condition,

iii) confirming said correlation of said blood coagulation activity marker in said body fluid sample determined in step i) to said clinical condition by diagnosing said clinical condition,

iv) based on the diagnosis of step iii), treating said clinical condition in said human or animal body.

The invention also pertains to a method for treating a clinical condition in a human or animal body by therapy or surgery, said method comprising the steps of

i) determining the blood coagulation activity of an individual according to a method of the present invention,

ii) correlating said blood coagulation activity of said individual determined in step i) to said clinical condition,

iii) confirming said correlation of said blood coagulation activity of said individual determined in step i) to said clinical condition by diagnosing said clinical condition,

iv) based on the diagnosis of step iii), treating said clinical condition in said human or animal body.

In a further aspect there is provided a diagnostic method practised on the human or animal body, said method comprising the steps of

i) determining the amount of at least one blood coagulation activity marker comprised in a body fluid sample according to a method of the present invention,

ii) correlating said amount of said blood coagulation activity marker in said body fluid sample determined in step i) to said clinical condition,

iii) based on the correlation of step ii), diagnosing said clinical condition in said human or animal body.

In a still further aspect there is provided a diagnostic method practised on the human or animal body, said method comprising the steps of

i) determining the blood coagulation activity of an individual according to a method of the present invention,

ii) correlating said blood coagulation activity of said individual determined in step i) to said clinical condition, and

iii) based on the correlation of step ii), diagnosing said clinical condition in said human or animal body.

In yet another aspect there is provided a kit of parts comprising means for detection and quantification of at least one blood coagulation activity marker present in a body fluid sample, and information linking said determined amount of said blood coagulation activity marker to the blood coagulation activity of an individual.

In a still further aspect the invention pertains to a kit of parts according to the invention for use in any of the methods of the present invention.

### DRAWINGS

FIG. 1 shows a plot of concentration of F₁₊₂ in morning urine vs. 24 h urine.

FIG. 2 shows a schematic presentation of a dipstick according to the invention.

FIG. 3a shows a photo of a dipstick with a negative result and FIG. 3b shows a photo of a dipstick with a positive result.

### DEFINITIONS

Blood coagulation activity shall be understood to comprise the overall biological activity resulting in blood coagulation, such as may be defined by the clotting assay as discussed above.

Reporter species shall be understood to comprise any species comprising at least one targeting species and at least one detectable label molecule, capable of being detected either directly or indirectly.

Target species shall be understood to comprise any species, preferably an antibody, that is able to specifically interact with another species which could be another targeting species or a blood coagulation marker to be determined and/or analysed.

F₁₊₂ is used synonymously with the terms “Prothrombin fragment 1+2” and “Fragment 1+2”.

### DETAILED DESCRIPTION OF THE INVENTION

Interrelationship between claimed methods

In one aspect the invention is directed to a method for correlating a known amount of at least one blood coagulation marker with a quantifiable “reporter species”. This method is used when it is initially required to provide a standard curve for a particular marker to be used in the assessment of the blood coagulation activity of an individual. Accordingly, the standard curve generated by this method is used in the method for determining an unknown amount or concentration of at least one blood coagulation activity marker.

The method for determining an unknown amount or concentration of at least one blood coagulation activity marker is carried out e.g. by a patient in his own home during a post-surgery phase in order to monitor the patients blood coagulation activity, and the obtained or recorded result may optionally be transmitted by any state of the art means of transmission to a health care unit for further analysis or evaluation. The methods for determining an unknown amount or concentration of at least one blood coagulation activity marker can be used for performing the correlation of the blood coagulation activity of an individual with the amount or concentration of the blood coagulation activity marker contained in a body fluid sample.

Accordingly, there is also provided a method for correlating the blood coagulation activity of an individual with the amount or concentration of a marker being present in a body fluid sample. This method is an essential requirement for being able to use the results generated by the method for determining an unknown amount or concentration of at least one blood coagulation activity marker in a method for determining the blood coagulation activity of an individual.

The method for correlating the blood coagulation activity of an individual with the amount or concentration of at least one blood coagulation activity marker being present in a body fluid sample may preferably comprise a reference to biological species being correlatable to the blood coagulation activity.

The method for determining the blood coagulation activity of an individual comprises—in preferred embodiments—at least one of the above-mentioned methods for determining at least one blood coagulation activity marker and subsequently correlating said determination of said marker with the blood coagulation activity of the individual in question

Consequently, in more preferred embodiments of the invention, the method for determining the amount of a marker in a body fluid sample preferably employ data obtainable by the method for correlating a predetermined amount of at least one blood coagulation activity marker with the amount of at least one quantifiably detectable reporter species.

The method for correlating the blood coagulation activity of a blood sample obtained from an individual with the amount of at least one blood coagulation activity marker comprised in a body fluid sample preferably comprises determining the amount of said at least one blood coagulation activity marker by the method as described herein above. The determination of said amount of said blood coagulation activity marker is preferably obtainable by the method for correlating a predetermined amount of at least one blood coagulation activity marker with the amount of at least one quantifiably detectable reporter species.

The method for correlating the blood coagulation activity of a blood sample obtained from an individual with the amount of at least one blood coagulation activity marker comprised in a body fluid sample obtained from said individual comprises in one embodiment correlating i) the blood coagulation time determined by means of a state of the art assay with ii) the determined amount of the at least one blood coagulation activity marker comprised in said body fluid sample. The determination of said at least one blood coagulation activity marker comprised in said body fluid sample is preferably obtainable by the method as described herein above. The determination of the amount of said blood coagulation activity marker is preferably obtainable by the method for correlating a predetermined amount of at least one blood coagulation activity marker with the amount of at least one quantifiably detectable reporter species.

The method for determining the blood coagulation activity of an individual preferably comprises correlating the determined amount of said at least one blood coagulation activity marker present in said body fluid sample with said blood coagulation activity of said individual by the method for correlating the blood coagulation activity of a blood sample with the amount of at least one blood coagulation activity marker comprised in a body fluid sample as described herein above. The method for correlating the blood coagulation activity of a blood sample obtained from an individual with the amount of at least one blood coagulation activity marker comprised in a body fluid sample preferably comprises determining the amount of said at least one blood coagulation activity marker by the method as described herein above, The determination of said amount of said blood coagulation activity marker is preferably obtainable by the method for correlating a predetermined amount of at least one blood coagulation activity marker with the amount of at least one quantifiably detectable reporter species.

The method for monitoring the blood coagulation activity of an individual preferably comprises obtaining a plurality of individual determinations of said blood coagulation activity of said individual by the methods described herein above.

The method of monitoring the clinical condition affecting the blood coagulation activity preferably comprises determining the amount of said at least one blood coagulation activity marker present in said plurality of body fluid samples obtained over a predetermined period of time by the methods described herein above The determination of the amount of the blood coagulation activity marker is preferably obtainable by the method for correlating a predetermined amount of at least one blood coagulation activity marker with the amount of at least one quantifiably detectable reporter species as described herein above.

In order to provide a significant correlation between blood coagulation activity and the concentration of the blood coagulation activity marker, the correlation is conducted between the concentration of a blood coagulation activity marker present in a body fluid sample and the concentration of a blood coagulation activity marker present in a blood sample from a given individual, when said body fluid sample and said blood sample is taken at approximately the same time The blood coagulation activity marker present in the blood sample should be a marker known to be a significant marker for the coagulation activity

The body fluid sample may be any sample easily obtained from the individual in question, for example a urine sample. A urine sample may be a spot urine sample, preferably taken from the morning urine hereafter designated morning urine, or it may be an average sample collected as 24 h urine samples hereafter designated 24 h urine sample.

The significant correlation may be determined by any suitable statistic method. In the present context the statistics are determined as Spearman rho correlation coefficient, a non-parametric correlation.

The Spearman rho correlation coefficient is preferably at least 0.3, such as at least 0.4, for example at least 0.42. Preferably, the Spearman rho correlation coefficient is at least 0.43. Most preferably, the Spearman rho correlation coefficient is at least approximately 0.459 for the correlation between a blood sample and a 24 h urine sample and at least approximately 0.438 for the correlation between a blood sample and a morning urine sample.

In another preferred embodiment there should be a highly significant correlation between the concentration of a blood coagulation activity marker present in a morning urine sample and the concentration of the same blood coagulation activity marker present 24 h urine samples from a given individual, when said morning urine sample and said 24 h urine samples are taken the same day.

Highly significant correlation within the present context means that the Spearman rho correlation coefficient is at least 0.5, preferably at least 0.6, more preferrably at least 0.7, even more preferably at least 0.8, yet more preferably at least 0.85, even more preferably at least 0.9. Most preferably, the Spearman rho correlation coefficient is approximately 0.907.

Blood coagulation markers

The present invention is not limited to the detection of any particular blood coagulation marker and the correlation of said marker With the blood coagulation activity of a patient. The present invention pertains to the detection of any blood coagulation marker capable of being detected in a body fluid sample in such a way that the detection is correlatable with blood coagulation activity.

Examples of suitable blood coagulation markers are markers selected from the group consisting of peptides comprising a fragment of pro-thrombin. The proportion of prothrombin is located at the amino-terminal end of the enzyme and consists of 271 amino acids according to Degen et al. (1983): Biochemistry vol. 22, p. 2087-2097). Hursting et al. (Clin. Chem., 1993, vol. 39(4), p. 583-591) have raised monoclonal antobodies against a fragment of pro-thrombin termed fragment 1+2 based on the amino acid sequence reported by Degen et al. (1983). In another study, Walz et al (Proc. Natl. Acad. Sci. USA, 1977, vol. 74(5), p. 1969-1972) reported that the pro-portion of pro-thrombin consists of 273 amino acids. Pelzer et al. (Thromb. Haemostas., 1991, vol. 65, p. 153-159) have raised monoclonal antobodies against fragment 1+2 of pro-thrombin based on the amino acid sequence reported by Walz et al. (1983). Hursting et al. (1993) attributed the difference between the sequences to two glutamic acids present in the C-terminal region of the pro-portion amino acid sequence (positions 266 and 267, respectively) reported by Walz et al. (1977).

Without being limited to one or the other of the sequences of the pro-portion of pro-thrombin referred to herein above, references below to amino acids of the pro-portion or pro-thrombin are based on the sequence reported by Degen et al. (1983).

The pro-portion contains two structurally similar, but functionally distinct domains termed pro-thrombin fragment 1 (amino acid residues 1 to 155) and pro-thrombin fragment 2 (amino acid residues 156 to 271). Conversion of pro-thrombin to thrombin initially results in the formation of a single pro-fragment, pro-thrombin fragment 1+2, (amino acid residues 1 to 271) Pro-thrombin fragment 1 and pro-thrombin fragment 2 are formed when pro-thrombin fragment 1+2 is further processed. Whereas pro-thrombin fragment 1 and fragment 2 are secreted in the urine, Bezeaud and Guillin (British J. Haematology, 1984, vol. 58, p. 597-606) did not detect pro-thrombin fragment 1+2 in analysed urine samples.

The blood coagulation activity marker is preferably selected from the group consisting of peptides comprising pro-thrombin Fragment 1+2 (F₁₊₂), peptides comprising pro-thrombin Fragment 1 (F₁), and peptides comprising pro-thrombin Fragment 2 (F₂). More preferred, the marker is selected from peptides comprising pro-thrombin Fragment 1+2 (F₁₊₂), from peptides comprising pro-thrombin Fragment 1 (F₁), and from peptides comprising pro-thrombin Fragment 2 (F₂).

In an even more preferred embodiment, the marker is selected from the group consisting of pro-thrombin Fragment 1+2 (F₁₊₂), pro-thrombin Fragment 1 (Fi), and pro-thrombin Fragment 2 (F₂) Also, the marker may essentially consist of either pro-thrombin Fragment 1+2 (F₁+₂), pro-thrombin Fragment 1 (F₁) or pro-thrombin Fragment 2 (F₂). Most preferably the marker is pro-thrombin Fragment 1+2.

In another embodiment the marker is pro-thrombin Fragment 1+2 (F₁₊₂ comprising amino acid residues I to 271 of pro-thrombin (Degen et al, 1983, ibid), including any functional variant thereof being at least 90%, such as at least 91%, for example at least 92%, such as at least 93%, for example at least 94%, such as at least 95%, for example at least 96%, such as at least 97%, for example at least 98%, such as at least 99% identical to pro-thrombin Fragment 1+2 (F₁₊₂) comprising amino acid residues 1 to 271 of pro-thrombin (Degen et al., 1983, ibid), said variant being obtained by deletion, insertion or substitution of at least one amino acid. Functional variants are identified by reaction with an antibody, preferably a monoclonal antibody, capable of detecting pro-thrombin Fragment 1+2 (F₁₊₂) comprising amino acid residues I to 271 of pro-thrombin (Degan et al., 1983, ibid), or part thereof, or identified by an antibody preferably a monoclonal antibody, capable of detecting pro-thrombin Fragment 1+2 (F₁₊₂) comprising amino acid residues 1 to 273 of pro-thrombin (Walz et al,, 1977, ibid), or part thereof.

The marker may also be pro-thrombin Fragment I (F₁) comprising amino acid residues 1 to 155 (Degen et al. (1983), ibid; Hursting et al. (1993), ibid) of pro-thrombin, including any functional variant thereof being at least 90%, such as at [least 91%, for example at least 92%, such as at least 93%, for example at least 94%, such as at feast 95%, for example at least 96%, such as at least 97%, for example at least 98%, such as at least 99% identical to pro-thrombin Fragment 1 (F₁) comprising amino acid residues 1 to 155 (Degen et al. (1983), ibid:, Hursting et al. (1993), ibid.), said functional variant being obtained by deletion, insertion or substitution of at least one amino acid. Functional variants are identified by reaction with an antibody, preferably a monoclonal antibody, capable of detecting pro-thrombin Fragment 1 (F₁) comprising amino acid residues 1 to 155 of pro-thrombin (Degen et al., 1983, ibid; Hursting et al (1993), ibid.), or part thereof.

The marker may also be prothrombin Fragment 2 (F₂) comprising amino acid residues 156 to 271 (Degen et al. (1983), ibid; Hursting et al. (1993), ibid.) of pro-thrombin, including any functional variant thereof being at least 90%, such as at least 91%, for example at least 92%, such as at least 93% for example at least 94%, such as at least 95%, for example at least 96%, such as at least 97%, for example at least 98%, such as at least 99% identical to prothrombin Fragment 2 (F₂) comprising amino acid residues 156 to 271 of prothrombin (Degen et al., 1983, ibid.; Hursting et al. (1993), ibid.). said variant being obtained by deletion, insertion or substitution of at least one amino acid. Functional variants are identified by reaction with an antibody, preferably a monoclonal antibody, capable of detecting pro-thrombin Fragment 2 (F₂) comprising amino acid residues 156 to 271 of pro-thrombin (Degen et al., 1983, ibid.; Hursting et al. (1993), ibid.), or part thereof, or identified by an antibody, preferably a monoclonal antibody, capable of detecting pro-thrombin Fragment 2 (F₂) comprising amino acid residues 156 to 273 of pro-thrombin Walz et al., 1977, ibid), or part thereof.

In a particularly preferred embodiment of the invention, the marker is detectable by a reporter species capable of detecting any of pro-thrombin Fragment 1+2 (F₁₊₂), pro-thrombin Fragment 1 (FI), and pro-thrombin Fragment 2 (F₂), or capable of detecting two or more of the fragments.

It is also possible to employ more than one reporter species for the detection of one or more blood coagulation activity markers present in a body fluid sample. In one embodiment, there is provided a first reporter species and a second reporter speices capable of detecting prothrombin Fragment 1+2 (F₁₊₂) and pro-thrombin Fragment 1 (F₁), respectively, pro-thrombin Fragment 1 (F₁₊₂) and pro-thrombin Fragment 2 (F₂), respectively, and pro-thrombin Fragment 1 (F₁₊₂) and pro-thrombin Fragment 2 (F₂), respectively, including functional variants as defined herein above.

In another embodiment, the blood coagulation activity marker is selected from the group consisting of peptides comprising a fragment of fibrinogen, such as the group consisting of peptides comprising fibrinopeptide A (FpA). In one embodiment the marker essentially consists of fibrinopeptide A (FpA), and in another embodiment the marker is fibrinopeptide A (FpA). In a preferred embodiment the marker is detectable by a reporter species capable of detecting fibrinopeptide A (FpA).

In a further embodiment the marker is selected from the group consisting of peptides comprising the carboxy-terminal 17 amino acid residues of the heavy chain of Factor Xₐ. Accordingly, the marker may essentially consist of the carboxy-terminal 17 amino acid residues of the heavy chain of Factor Xₐ, or the marker may be the carboxy-terminal 17 residues of the heavy chain of Factor Xₐ.

Assays for detection of a blood coagulation marker in a body fluid sample

The present invention does not depend on any particular type of assay for the detection of the blood coagulation marker in a body fluid sample. Any assay capable of detecting a blood coagulation activity marker in a body fluid sample can be used in conjunction with the present invention. Assays based on a specific recognition of the marker are preferred, such as qualitative and/or quantitative assays involving the use of immunoreactive species, i.e. antigens, haptens and antibodies or fragments thereof.

The present invention may in one embodiment employ standard immunohistochemical or cytochemical detection procedures, or suitable modifications thereof, for the detection of the blood coagulation marker according to the invention. Accordingly, the invention may employ any assay resulting in the recognition of an antigenic determinant mediated by an immunochemical reaction of the antigenic determinant with a specific so-called primary antibody capable of reacting exclusively with the target antigens determinant in the form of a blood coagulation activity marker.

The primary antibody is preferably labelled with an appropriate label capable of generating—directly or indirectly—a detectable signal. The label is preferably an enzyme, a radioactive isotope, a fluorescent group, a dye, a chemiluminescent molecule and a heavy metal such as gold

In another embodiment, the invention employ the detection of the primary antibody by immunochemical reaction with specific so-called secondary antibodies capable of reacting specifically with the primary antibodies. In this case the secondary antibodies are preferably labelled with an appropriate label such as an enzyme, a radioactive isotope, a fluorescent group, a dye, a chemiluminescent molecule or a heavy metal such as gold.

In yet another embodiment, the present invention employs a so-called linker antibody as a means of detection of the marker. This embodiment exploits that the immunochemical reaction between the target antigenic determinant in the form of the marker and the primary antibody is mediated by another immunochemical reaction involving the specific linker antibody capable of reacting simultaneously with both the primary antibody as well as another antibody to which enzymes have been attached via an immunochemical reaction, or via covalent coupling and the like.

In yet another embodiment according to the present invention, the immunochemical reaction between the target antigenic determinant in the form of the marker and the primary antibody, or alternatively, between the primary antibody and the secondary antibody, is detected by means of a binding of pairs of complementary molecules other than antigens and antibodies. A complementary pair such as e.g. biotin and streptavidin is preferred. In this embodiment, one member of the complementary pair is attached to the primary or secondary antibody, and the other member of the complementory pair is contacted by any suitable label such as e.g. an enzymes, aradioactiveisotope, a fluorescent group, a dye or a heavy metal such as gold.

A body fluid sample is preferably brought into contact with a carrier and optionally treated with various chemicals to facilitate the subsequent immunochemical reactions. The body fluid sample contacting the carrier is referred to as a specimen. The body fluid sample in one preferred embodiment is then subjected to treatment with a labelled or non-labelled primary antibody, as appropriate, whereupon the antibody becomes immunochemically bound to the blood coagulation activity marker comprised in the sample. After removal of excess antibody by suitable washing of the specimen comprising the body fluid sample composition, the antibody bound to the blood coagulation activity marker is detected by reaction with appropriate reagents, depending on the choice of detection system.

After removing excess labelled reagent from the chosen detection system, the specimen comprising the blood coagulation activity marker to be detected and optionally also quantified is preferably subjected to at least one of the detection reactions described below. The choice of detection reaction is influenced by the marker in question as well as by the label it is decided to use.

When an enzyme label is used, the specimen is treated with a substrate, preferably a colour developing reagent. The enzyme reacts with the substrate, and this in turn leads to the formation of a coloured, insoluble deposit at and around the location of the enzyme. The formation of a colour reaction is a positive indication of the presence of the marker in the specimen.

When a heavy metal label such as gold is used, the specimen is preferably treated with a so-called enhancer in the form of a reagent containing e.g. silver or a similar contrasting indicator, Silver metal is preferably precipitated as a black deposit at and around the location of the gold

When a fluorescent label is used, a developing reagent is normally not needed.

After at least one washing step, some of the constituents of the specimen are preferably coloured by reaction with a suitable dye resulting in a desirable contrast to the colour provided by the label in question. After a final washing step, the specimen is preferably coated with a transparent reagent to ensure a permanent record for the examination.

Detection of the label in question preferably indicate both the localization and the amount of the target antigenic determinant in the form of the blood coagulation activity marker. The detection may be performed by visual inspection, by light microscopic examination in the case of enzyme labels, by light or electron microscopic examination in the case of heavy metal labels, by fluorescence microscopic examination, using irradiated light of a suitable wavelength, in the case of fluorescent labels, and by autoradiography in the case of an isotope label. Detection of the presence of the marker—and preferably also the amount of the marker—by visual inspection of the specimen is preferred.

In a particularly preferred embodiment, the visual detection is based on a cut-off point above which one visible colour indicates the presence of the marker above a certain minimum amount (cutoff point), and below Which cut-off point another visible colour or no colour change indicates that the marker is present in an amount of less than that indicated by the cut-off point The visual colour may be in any suitable form, such as in the form of a spot, a line, a cross, a triangle, a square, a circle, preferably the colour is in the form of a spot or a line, most preferably in the form of a line.

More preferably the method and system includes a control system as well, for example in the form of a control change in colour somewhere in the system to indicate that the test has been conducted correct although the test is negative, i.e. no value above the cut-off value is shown. Preferably, such control system involves a change in colour based on the presence of rhodamine.

The method and system according to the present invention provides a possibility of adjusting (fine-tuning) the cut-off point at any suitable value. For most purposes, the cut-off point is at least 0.1 nM, for example at least 0.15 nM, such as at least 0.20 nM, for example at least 0.25 nM, such as at least 0.30 nM. In another preferred embodiment the cutt-off point is between 0.1 and 2.0 nM, for example between 0.20 and 1.5 nM, such as between 0.30 and 1.0 nM. Most preferably the cut-off point is around 0.30 nM.

If the blood coagulation marker to be determined is Prothrombin Fragment 1+2 and/or pro-thrombin Fragment 1 (F₁) and/or pro-thrombin Fragment 2 (F₂), the cut-off point is preferably between 0.1 and 2.0 nmol/L, more preferably between 0.20 and 1.5 nmol/L, yet more preferably between 0.3 and 1.0 nmol/L, even more preferably, between 0.3 and 0.8 nmol/L, yet more preferably, between 0.3 and 0.5 nmol/L, even more preferably, between 0.3 and 0.4 nmol/L, most preferably around 0.30 nmol/L.

It is contained within the present invention to use more than one cut-off point within the same assay, such as two cut-off points, for example 3 cut-off points, such as 4 cut-off points, for example 5 cutoff points, such as more than 5 cut-off points within the same assay. An assay using several different cut-off values would allow determination of the amount of blood coagulation marker to a defined interval.

Enzyme-Linked Immuno-Sorbent Assays (ELISA) in which an antigen, hapten or antibody is detected by means of an enzyme which is linked such as covalently coupled or conjugated either—when an antigen or hapten is to be determined—to an antibody which is specific for the antigen or hapten in question, or—when an antibody is to be determined—to an antibody which is specific for the antibody in question—may be used for detecting the blood coagulation activity marker according to the present invention, in particlar in relation to microfluid systems (see herein below).

In one preferred embodiment, the blood coagulation activity marker to be detected is bound or immobilized by immunochemically contacting the marker with a so-called “catching” antibody attached by e.g. non-covalent adsorption to the surface of an appropriate material. Examples of such materials are polymers such as e.g. nitrocellulose or polystyrene, optionally in the form of a stick, a test strip, a bead or a microtiter tray. A suitable enzyme-linked specific antibody is allowed to bind to the immobilized marker to be detected. The amount of bound specific antibody, i.e. a parameter that is correlatable to the immobilized marker, is determined by adding a substance capable of acting as a substrate for the linked enzyme. Enzymatic catalysis of the substrate results in the development of a detectable signal such as e.g. a characteristic colour or a source of electromagnetic radiation. The intensity of the emitted radiation can be measured e.g. by spectrophotometry, by colorimetry, or by comparimetry. The determined intensity of the emitted radiation is correlateble—and preferably proportional—to the quantity of the blood coagulation activity marker to be determined. Examples of preferred enzymes for use in assays of this type are e.g. peroxidases such as horseradish peroxidase, alkaline phosphatase, glucose oxidases, galactosidases and ureases.

It is one objective of the present invention, to provide methods for determining the amount of at least one blood coagulation activity marker using a lateral flow test type of assay involving for example a dipstick, a syringe, a tube or a container. Such assays involve immobilisation of the blood coagulation activity marker(s) on an extended solid phase using a targeting species, preferably an antibody. The extended solid phase used in the present invention may be employed in a variety of forms or structures. The extended solid phase has a location where the targeting species can bind or associate, and the formation of such an extended solid phase with said targeting species, preferably an antibody, enables contacting a sample and other materials used in the method of the invention. Preferred samples are body fluid samples, such as a urine sample.

Preferably, the extended solid phase is formed in a way which enables simple manipulation for easy contact with the sample and other reagents

The samples and other reagents can be drawn in and ejected from a syringe, caused to flow through a tube, or deposited in a container such as a test tube shaped container. In such devices, the extended solid surface can form the whole of the device, or part of it, where, in the case of a syringe, tube or container, the part formed of the extended solid surface will at least be exposed at the inside of the device to permit contact with samples and reagents. Targeting species, preferably an antibody, are preferably concentrated at one location of the extended solid surface, to be exposed to the sample. Preferably, the targeting species is immobilised on the solid surface.

In one preferred embodiment the solid surface is comprised within a lateral flow device. In another preferred embodiment the solid surface is a dipstick or part thereof. In particular such solid surface, which is a dipstick or part thereof is made of nitrocellulose.

In one preferred embodiment of the present invention the lateral flow device is a dipstick. Preferably, in such a dipstick the extended solid phase is included at at least one end, and the targeting species, preferably antibodies, that are bound to or associated with the extended solid phase are concentrated at the end of the dipstick. Preferably, the extended solid phase comprise the entire dipstick, with the targeting species, preferably an antibody, concentrated at one end, or in more than one location.

The dipstick of the present invention may be entirely formed from the extended solid surface, at one end of which has been conjugated a coating of targeting species, preferably an antibody In another embodiment the dipstick has an extended solid phase one end of which is adhered to a body portion. A coating of targeting species, preferably an antibody, is conjugated to the extended solid phase. In yet another embodiment the extended solid phase entirely forms a tubular container into which a sample can be placed. Coatings of targeting species, preferably an antibody, are located near the bottom of the container and are concentrated in one or more locations.

The extended solid phase is composed of any material onto which the desired targeting species, preferably an antibody, can be effectively bound. For covalent binding with antibody protein, the solid phase material can be chosen to contain a functional carboxyl surface, with use of a water-soluble carbodiimide as a conjugation reagent. A preferred material is acrylic resin, which has a carboxylated surface that enables binding the desired targeting species, preferably an antibody, by conjugation. For materials with amino surface groups, reactive carboxyl intermediates can be prepared by reacting with succinic anhydride. A variety of inorganic supports, typically glass, can also be prepared for covalent coupling with targeting species, preferably an antibody,. Reference is made, for example, to “Enzymology, A Series of Textbooks and Monographs,” Vol. 1, Chapter 1, 1975, the disclosure of which is incorporated herein by reference.

In one preferred embodiment the extended solid phase is a nitrocellulose membrane

Extended solid phase materials capable of binding targeting species, preferably an antibody, are selected from materials which do not cause serious interference with the assay steps.

For convenience in the following description, the extended solid phase will be referred to as the preferred dipstick, although other forms may be used as explained herein above.

In accordance with the method of the present invention, the antibody targeting species recognising blood coagulation markers, are derived from the 1 g fraction of an antiserum or from monoclonal antibodies. Such targeting species can be bound to or associated with respectively an extended solid phase dipstick and they can bound to or associated with a polymeric carrier molecule comprised within a mobile reporter species. Coupling techniques between the antibody protein and various solid phase materials or polymeric carrier molecules are well developed (see, for example U.S. Pat. No. 3,853,987).

In one preferred embodiment of the present invention the polymeric carrier molecule has a hydrophilic sugar chain backbone. More preferably, the conjugate has a polymeric dextran backbone.

In one preferred embodiment of the present invention the reporter species comprise a polymeric carrier molecule as described in detail herein below. Furthermore, the reporter species of the present invention preferably comprise one or more targeting species. Furthermore, the reporter species of the present invention comprises at least one labelling species. Such labeling species could be selected from the group consisting of; coloured dye molecules, enzymes, fluorescent molecules, chemiluminescent molecule, radioactive isotopes, metal elements or iron oxide in order to provide X-ray fluorescent or electromagnetic signals.

Preferably coloured dye molecules should be visible on the solid support under assay conditions, allowing direct determination without instrumentation. Preferably, coloured dye molecules have an intense colour which for example could be red, blue, yellow, orange, green or any other colour. More preferably, the polymeric carrier molecules according to the invention include any coloured dye molecule which can be detected by direct visual observation. Most preferably, the coloured dye is rhodamine.

In one embodiment the reporter species preferably comprises a polymeric carrier molecule that can bind at least 10, such as at least 20, for example at least 40, such as at least 60, for example at least 80 labelling molecules. In another embodiment the reporter species preferbably comprises a polymeric carrier molecule that can bind at least 2 targeting species molecules, such as at least 5 targeting species molecules, for example at least 10 targeting species molecules, such as at least 15 targeting species molecules.

In one embodiment of the method of the present invention described above, the resulting immunocomplex is a multilayered “sandwich” comprising;

Extended solid phase dipstick+first targeting species, preferably an antibody+blood coagulation marker+reporter species, preferably comprising at least one polymeric carrier molecule, at least one second targeting species and at least one labelling species.

The amount of antibody required for covalent binding, however, can be less than a thousand times that of passive adsorption to a plastic such as polyvinyl chloride and the economics of using such an amount of highly specific targeting species, preferably an antibody, can be prohibitive.

An alternative way of binding that retains some strength of the covalent binding as well as the specificity of targeting species, preferably an antibody, is to bridge the targeting species and the solid phase with a first antibody, an antispecies antibody targeted against the Fc portion of the targeting antibody. Such an Fc portion is illustrated e.g. in “Immunology” (1981), The Upjohn Company, Kalamazoo, Mich.

That is, an inexpensive first antibody may initially be covalently bound to the solid phase, and the bound first antibody attracts the species-specific Fc portion of a targeting antibody, leaving the functional epitope of the targeting antibody unaltered with regard to an antigen of a blood coagulation marker. Bridged with such a first antispecies antibody, the immunoassay of the present invention brings about the following coupling “sandwich” in the case of detection of a viral species:

Extended solid phase dipstck+antispecies antibody+targeting antibody+viral antigen+targeting antibody+reporter species comprising at least one antispecies antibody, at least one polymeric carrier molecule and at least one labelling species.

In the direct binding assay of the present invention, the couplings between the extended solid phase and targeting species, preferably an antibody, as well as the couplings between the individual species of the reporter species including polymeric carrier molecules according to the invention, at least one targeting species, preferably an antibody, and labelling species of the reporter species, are preferably prepared in advance.

In one preferred embodiment of the present invention the detection of attached reporter species on a dipstick, is made independent of immune chemistry, in order to use a minimal amount of wet chemistry. Instead, concentration of the targeting species, preferably an antibody, to one location of the dipstick, results in that the bound reporter species according to the invention also are concentrated at one location. In case the labelling molecule is a coloured dye molecule, such concentration could enable direct visual detection.

In one preferred embodiment of the present invention the test is performed as a one-step test. Couplings between the extended solid phase and reporter species, preferably an antibody, as well as couplings between the polymeric carrier molecules according to the invention and reporter species, preferably an antibody, are prepared in advance. Furthermore, the polymeric carrier molecules are comprised within the lateral flow device. Hence, the sole step remaining to be performed is to apply a body fluid sample, such as a urine sample, directly to the lateral flow test after which the test results appear, for example as a concentration of coloured dye molecules, which can be observed by visual inspection.

In one embodiment, the present method employs a direct binding assay instead of a competitive binding assay where a dynamic equilibrium necessitates lengthy incubation. The disclosed method can, of course, be employed in a competitive protein binding assay as well. The roles of the immune analytes antibody and antigen can also be interchanged, still making use of the immobilized solid phase for the signal amplification. Binding of antibody or various antigen molecules to the solid phase matter is well known, in passive adsorption as well as in covalent coupling.

The method of the invention can also be designed to assay several analytes in a single procedure where each analyte is represented by a particular pair of corresponding binding partners including antibodies, antigens, and the same or different polymeric carrier molecules comprising one or more reporter species.

Detection of different types of blood coagulation markers can be done in accordance With the invention by conjugating a plurality of different targeting species, preferably antibodies, capable of forming complexes with different blood coagulation markers, to the extended solid phase and to the reporter species. The detection of bound material as described above following the assay indicates that one or more of the different blood coagulation markers are present in the specimen, and this assay, if positive, can be followed by assays for individual blood coagulation markers selcted from the ones which were tested for simultaneously. Immunochemical assays of a type analogous to ELISA but employing other means of detection are also suitable for detecting the marker according to the present invention. Such assays are typically based on the use of specific antibodies to which fluorescent or luminescent marker molecules are covalently attached. So-called “time-resolved fluorescence” assays are particularly preferred and typically employ an europium ion label or an europium chelator, even though certain other lanthanide species or lanthanide chelators may also be employed. In contrast to many traditional fluorescent marker species the fluorescence lifetime of lanthanide chelates is generally in the range of 100-1000 microseconds. In comparison, fluorescein has a fluorescence lifetime of only about 100 nanoseconds or less. By making use of a pulsed light source and a time-gated-fluorometer, the fluorescence of lanthanide chelate compounds can be measured in a time-window of about 200-600 microseconds after each excitation. A main advantage of this technique is the reduction of background signals which may arise from more short-lived fluorescence of other substances present in the analysis sample or in the measurement system.

It is another object of the present invention to detect blood coagulation markers in a body fluid sample by means of miniaturized, integrated microfluid devices and systems incorporating such devices.

In a microfluid device it is possible to perform a series of defined operations in very small amount of solution, preferably within microliter range (SKAL DER TAL PA?). For example such devices can integrate all operations involved in sample acquisition and storage, sample preparation and several steps of sample analysis in a single miniaturised integrated unit.

In one preferred embodiment of the present invention, at least one blood coagulation marker is detected in at least one body fluid sample by an immunochemical reaction as described herein above within a microfluid device.

The microfluid devices used with the present invention will typically be one component of a larger diagnostic system which further preferably includes a reader device for scanning and obtaining data from the device, and a computer based interface for controlling the device and/or interpretation of the data derived from the device.

A suitable microfluid device should comprise at least one compartment chamber. The at least one compartment chamber comprises one or more of the following i) at least one or a plurality of first target species, preferably antibodies and ii) at least one or a plurality of second target species, preferably antibodies.

However, a suitable microfluid device could comprise more than one compartment chamber comprising similar or distinct first and/or second target species and/or reporter species.

The first target species, preferably antibodies, could be derived from any source known to a person skilled in the art and they should interact specifically with the blood coagulation marker to be determined. Reporter species preferably comprise second target species, preferably antibodies, which could be derived from any source known to a person skilled in the art and they should interact with the first target species.

Preferably, each or a plurality of target species are bound or coupled to or immobilised on a suitable solid support. In one preferred embodiment such solid support is the walls and/or surfaces and/or part of said chamber. Suitable solid supports include those that are well known in the art, e.g., agarose, cellulose, glass, silica, divinylbenzene, polystyrene, etc.

In one embodiment of the present invention the at least one or a plurality of reporter species comprise a polymeric carrier molecule. In one preferred embodiment of the present invention the polymeric carrier molecule has a hydrophilic sugar chain backbone as described in detail herein below.

In a preferred embodiment of the present invention at least one or a plurality of said reporter species, or a subset thereof, comprises one or more appropriate labels capable of generating—directly or indirectly—a detectable signal.

In another preferred embodiment the polymeric carrier molecule comprises at least one or more appropriate labels capable of generating—directly or indirectly—a detectable signal.

The label is preferably an enzyme, biotin, a radioactive isotope, a fluorescent group, a dye, a chemiluminescent molecule and a heavy metal such as gold, as describe herein above.

In one preferred embodiment an array of ordered target species and/or reporter species are comprised on a microchip. This would allow determination of the presence of a multitude blood coagulation markers within the same assay or determination of one or more blood coagulation markers in combination with different analytes within the same assay.

In one preferred embodiment the microfluid device comprise the following;

I. A microchip comprising an immobilised defined array of ordered targeting species, preferably antibodies, recognising different analytes, bound to or associated therewith.

II. A mixture of reporter species in solution, each comprising one kind of targeting species, such as every targeting species on the microchip is comprised within at least one reporter species. The reporter species should further comprise at least one labelling species and optionally a polymeric carrier molecule.

Such microchip and such mixture of reporter species could be comprised within the same reaction chamber or they could be comprised in distinct reaction chambers within the microfluid device. The body fluid sample could be exposed to the reporter species prior to, simultaneous with or following exposure to the microchip.

In addition to the various reaction chambers, the device will generally comprise a series of fluid channels, which allow for the transportation of the sample, or a portion thereof, among the various reaction chambers. Further chambers and components may also be included to provide reagents, buffers, sample manipulation, e.g., mixing, pumping, fluid direction (i.e., valves) heating and the like.

The sample collection portion of the device of the present invention preferably provides for the identification or nummeration of individual samples, while preventing contamination of samples by each other, external elements, or contamination of a working environment or an external environment by the sample. In a preferred embodiment more than one sample can be analysed at a given time within the microfluid device.

Typically, the sample(s) are applied by directly injecting the sample(s) into the sample collection chamber(s) through a sealable opening, e.g., an injection valve, or a septum. Generally, sealable valves are preferred to reduce any potential threat of leakage during or after sample injection. Alternatively, the device may be provided with a hypodermic needle integrated within the device and connected to the sample collection chamber, for direct acquisition of the sample into the sample chamber. This can substantially reduce the opportunity for contamination of the sample.

Reagents, which for example could be reporter species or targeting species, preferably antibodies, may generally be stored within the sample collection chamber of the device or may be stored within a separately accessible chamber, wherein the reagents may be added to or mixed with the sample upon introduction of the sample into the device. These reagents may be incorporated within the device in either liquid or lyophilized form, depending upon the nature and stability of the particular reagent used.

Gathering data from the analysis operations is carried out using any method known to a person skilled in the art. For example, the microchips may be scanned using lasers to excite fluorescent labels bound to reporter species and/or polymeric carrier molecules bound to specific regions of the microchip, which can then be imaged using charged coupled devices (“CCDs”) for a wide field scanning of the microchip. Alternatively, another particularly useful method for gathering data from the microchip is through the use of laser confocal microscopy which combines the ease and speed of a readily automated process with high resolution detection. Particularly preferred scanning devices are generally described in, e.g., U.S. Pat. Nos. 5,143,854 and 5,424,186.

Following the data gathering operation, the data will typically be reported to a data analysis operation. To facilitate the sample analysis operation, the data obtained by the reader from the device will typically be analyzed using a digital computer. Typically, the computer will be appropriately programmed for receipt and storage of the data from the device, as well as for analysis and reporting of the data gathered, i.e., interpreting fluorescence data to determine the qauantity of a specific blood coagulation marker with normalization of background.

As a miniaturized device, the body of the microfluid device as described herein will typically be approximately 1 to 20 cm in length by about 1 to 10 cm in width by about 0.1 cm to about 2 cm thick. Although indicative of a rectangular shape, it will be readily appreciated that the devices of the invention may be embodied in any number of shapes depending upon the particular need. Additionally, these dimensions will typically vary depending upon the number of analysis to be performed by the device, the complexity of these operations and the like. As a result, these dimensions are provided as a general indication of the size of the device.

The number and size of the reaction chambers included within the device will also vary depending upon the specific application for which the device is to be used. Generally, the device will include at least one reaction chamber, preferably at least two distinct reaction chambers, and preferably, at least three, four or five distinct reaction chambers, all integrated within a single body. Individual reaction chambers will also vary in size and shape according to the specific function of the reaction chamber

For example, in some cases, circular reaction chambers may be employed. Alternatively, elongate reaction chambers may be used. In general however, the reaction chambers will be from about 0.05 mm to about 20 mm in width or diameter, preferably from about 0.1 mm to about 2.0 mm in width or diameter and about 0.05 mm to about 5 mm deep, and preferably 0.05 mm to about 1 mm deep For elongate chambers, length will also typically vary along these same ranges.

Microfluid channels, on the other hand, are typically distinguished from chambers in having smaller dimensions relative to the chambers, and will typically range from about 10 μm to about 1000 μm wide, preferably, 100 μm to 500 μm wide and about 1 μm to 500 μm deep. Although described in terms of reaction chambers, it will be appreciated that these chambers may perform a number of varied functions, e.g., as storage chambers, incubation chambers, mixing chambers and the like

In some cases, a separate chamber or chambers may be used as volumetric chambers, e.g., to precisely measure fluid volumes for introduction into a subsequent reaction chamber.. In such cases, the volume of the chamber will be dictated by volumetric needs of a given reaction. Further, the device may be fabricated to include a range of volumetric chambers having varied, but known volumes or volume ratios (e.g., in comparison to a reaction chamber or other volumetric chambers).

In one embodiment wells manufactured into the surface of one planar member make up the various reaction chambers of the device. Channels manufactured into the surface of this or another planar member make up fluid channels which are used to fluidly connect the various reaction chambers. Another planar member is then placed over and bonded to the first, whereby the wells in the first planar member define cavities within the body of the device which cavities are the various reaction chambers of the device. Similarly, fluid channels manufactured in the surface of one planar member, when covered with a second planar member define fluid passages through the body of the device These planar members are bonded together or laminated to produce a fluid tight body of the device.

In some cases, the body of the microfluid device may include some parts of injection molded plastics, which for example could be polycarbonate, polystyrene, polypropylene, polyethylene, acrylic, and commercial polymers such as Kapton, Valox, Teflon, ABS, Delrin and the like, while other portions of the body may comprise etched silica or silicon planar members, and the like. For example, injection molding techniques may be used to form a number of discrete cavities in a planar surface which define the various reaction chambers, whereas additional components, e.g., fluid channels, microchips, etc, may be fabricated on a planar glass, silica or silicon chip or substrate. Lamination of one set of parts to the other will then result in the formation of the various reaction chambers, interconnected by the appropriate fluid channels.

The surfaces of the fluid channels and reaction chambers which contact the samples and reagents may also be modified to better accommodate a desired reaction. Surfaces may be made more hydrophobic or more hydrophilic depending upon the particular application. Alternatively, surfaces may be coated with any number of materials in order to make the overall system more compatible to the reactions being carried out.

Additional assays employing immunochemical detection techniques capable of being exploited in the present invention belong to the group of “immunoblotting” procedures, such as e.g. “dot blot” and “west em blot” procedures. In the western blot procedure, which is typically employed for the analysis and identification of antigenic polypeptides or proteins, the blood coagulation activity marker of interest is preferably separated by polyacrylamide gel electrophoresis and subsequently transferred by means of e.g. electrophoresis to membrane sheet such as e.g. a sheet of nitrocellulose or chemically treated paper to which the marker is capable of binding. An appropriate specific antibody is initially added and later followed by a labelled second antibody against the first antibody. Labelled protein-A may be added as an alternative to the addition of labelled second antibody. The label is preferably a radioisotope, a fluorescent dye, an enzyme or a heavy metal such as gold or a colloid thereof. The presence and location of the marker is detected in an appropriate manner as described herein above.

Preferred assays for detection of a blood coagulation marker in a body fluid sample The below-mentioned assays and detection procedures illustrate preferred methods for the detection and/or quantification of a blood coagulation activity marker according to the invention.

U.S. Pat. No. 4,703,017 relates to a solid phase assay for an analyte, wherein a binder is supported on a solid support, such as nitrocellulose, and the tracer is comprised of ligand labeled with a colored particulate label, such as a liposome including a dye. The assay has a high sensitivity, and the tracer is visible on the support under assay conditions, whereby tracer can be determined, without instrumentation, and without further treatment thereof.

Accordingly, the present invention in one aspect provides a method for detecting for a blood coagulation activity marker, said method comprising:

i) contacting

a) a reporter species comprising a first targeting species, preferably an antibody or a fragment thereof, with

b) a composition comprising

a body fluid sample comprising said marker, and

a reporter species comprising a second targeting species, preferably an antibody or a fragment thereof and a visible, particulate label in the form of a liposome or a microcapsule comprising a coloured particle in the form of a visable dye such as rhodamine,

wherein the second,.targeting species is capable of contacting either the first targeting species or the marker, whereby the visible label is brought into contact with either the first targeting species or the marker contacted by the second targeting species,

said reporter species being in contact with a solid test area, preferably nitrocellulose, or any other material having a surface capable of supporting an antibody in a concentration of at least 1 μg/cm², and

ii) determining the visibility of the tracer bound in said test area as a measure of the blood coagulation activity marker present in a sample

U.S. Pat. No. 4,952,517 in one aspect relates to an immunoassay procedure consisting of contacting a sample containing an analyte with a known amount of an antibody thereto and with a calibrated amount of the analyte itself that is conjugated to a solid support. When the level of the analyte in the sample exceeds a certain threshold level, the antibody will be insufficient to block all of the corresponding analyte on the solid support. Thus, upon addition of labelled antibody to the assay system, a detectable immunoreaction product becomes attached to the support to indicate that the amount of analyte in the sample exceeds the threshold level On the other hand, if the level of the analyte in the sample is below the threshold amount, the free antibody will be sufficient to block all of the corresponding analyte on the solid support preventing labelled antibody from forming a detectable immunoreaction product on the support and thus no signal will appear.

Accordingly, in one particularly interesting embodiment of the present invention there is provided an immunoassay procedure to determine the initial presence of at least a prespecified amount of a first blood coagulation activity marker present in a liquid sample, wherein said prespecified amount corresponds to a desired cut-off value, said procedure comprising the steps of;

i) establishing an immunochemical reaction phase by admixing a liquid sample containing an initially unknown amount of said first blood coagulation activity marker with

a) a known amount of a first reporter species that is specifically immunoreactive with said marker, and

b) a predetermined quantity of said first blood coagulation activity marker, or a second marker that has immunological reaction characteristics which are immunospecifically the same as the immunological reaction characteristics of said first marker,

wherein said known amount of said reporter species is immunochemically equivalent to the total of said prespecified amount of said first marker corresponding to the cut-off value and said predetermined quantity of said first or second marker cited in b) above,

whereby, when the initially unknown amount of the first blood coagulation activity marker in the liquid sample exceeds the prespecified amount, unreacted first or second marker will be available for further immunospecific reaction in the reaction phase,

ii) contacting the thus established reaction phase with a quantity of a second reporter species that has immunological reaction characteristics which are immunospecifically the same as the immunological reaction characteristics of said first reporter species, said second reporter species being quantifiably detectable; and

iii) determining the initial presence of more than said prespecified amount of first marker present in said sample by detecting the existence of a specific immunoreaction product containing said quantifiably detectable reporter species.

U.S. Pat. No. 5,610,077 relates to a method for carrying out a specific binding assay. Accordingly, in one aspect of the present invention there is provided a method for detecting or quantifying a blood coagulation activity marker, said method comprising the steps of

i) reacting

a) a body fluid sample comprising a blood coagulation activity marker, with

b) a reporter species comprising a first antibody specific for the marker, said reporter species being immobilised on a solid support, and

c) a reporter species comprising a second antibody, said second antibody being quantifiably detectable, said second antibody forming with said marker and said first antibody a sandwich complex by reaction between whatever quantities are present of the marker and the first antibody, and

ii) immobilising the second, quantifiably detectable antibody to the support via the marker, and

iii) detecting the second, quantifiably detectable antibody as an index of the quantity of the marker being tested for in the sample,

wherein said first and second antibody are reacted together prior to reaction with the body fluid sample, and wherein competitive interferences are avoided by preferably using monoclonal antibodies of narrow and different, non-interfering specificity, and wherein the reporter species comprising the first antibody is immobilised on the surface of a displacer body occupying a majority of the volume of a well or cup containing aqueous liquid in which the specific binding reaction takes place.

U.S. Pat. No. 5,521,102 relates to a controlled sensitivity immunochromatographic assay exploiting the binding of a predetermined amount of an analyte to an antibody in enabling the control of the assay sensitivity. A predetermined amount of an antibody is employed for binding an analyte present in the sample, up to a certain threshold amount. Analyte present in the sample at a level above the threshold amount proceeds unbound onto a membrane, where it reacts with an antibody-coated latex and a second, immobilized antibody to generate a positive signal.

Accordingly, the present invention in one embodiment pertains to a method for the detection of a blood coagulation activity marker in a body fluid sample, said method comprising the steps of:

i) providing a device comprising

a) a sample application area comprising a predetermined amount of a reporter species comprising an antibody capable of binding said marker deposited thereon, said area being in fluid communication with

b) a reaction zone comprising a mobilizable reporter species comprising an antibody capable of binding said marker, said reporter species further comprising at least one visually detectable particle, and

c) a detection zone comprising a reporter species comprising an antibody capable of binding said marker,

wherein, when said body fluid sample comprising said marker is applied to said sample application area, a threshold amount of the marker is bound to said antibody and thereby prevented from binding to the antibody being present in the reaction zone, and

wherein the marker remaining unbound in said body fluid sample passes from the sample application area through said reaction zone, where it is bound to said mobilizable reporter species comprising i) an antibody capable of binding said marker, and ii) at least one visually detectable particle and/or at least one fluorescently detectable particle, and

wherein the marker bound to the mobilizable reporter species is brought into contact with the detection zone, where the marker is bound to said reporter species comprising said antibody capable of binding said marker, and

wherein said binding of said marker results in immobilization of said mobilizable reporter species further comprising i) an antibody capable of binding said marker, and ii) at least one visually detectable particle and/or at least one fluorescently detectable particle,

ii) applying the body fluid sample to the sample application area of the device

iii) allowing the body fluid sample to traverse the sample application area, the reaction zone and the detection zone, and

iv) determining the presence and/or concentration of said analyte in the liquid sample based on the visually and/or fluorescently detectable signal generated in the detection zone.

U.S. Pat. No. 4,943,552 relates to a lateral flow method for assaying a sample for the presence and/or concentration of an analyte Accordingly, the present invention in one embodiment pertains to a method for determining the presence or approximate amount of a blood coagulation activity marker in a body fluid sample, said method comprising the steps of,

i) placing the body fluid sample on a sample application zone of a lateral flow membrane comprising

a) a liquid sample application zone, and

b) at least one indicator zone spaced apart from said application zone laterally on the surface of said membrane, said indicator zone further comprising immobilized thereto a reporter species capable of binding the marker,

wherein the application of said sample to the application zone results in said sample flowing laterally from said application zone through said indicator zone, and

wherein said flow results in the contacting of said marker and said reporter species immobilized onto the indicator zone, and

ii) assessing the binding of marker in the indicator zone to determine the presence, absence or approximate amount of analyte.

U.S. Pat. No. 4,642,285 relates to an immunoassay for the detection of an antigen in a body fluid Accordingly, the present invention in one embodiment pertains to reacting a first antibody in contact with a solid support such as nitrocellulose With a blood coagulation activity marker present in a body fluid sample. In a first incubation step, the immobilized reporter species comprising the first antibody is contacted by the marker in the body fluid sample. The antibody to which the marker is attached is washed and subsequently incubated with a reporter species comprising a second antibody tagged with a colour or an enzyme, During this second incubation step, the colour or enzyme tagged antibody reacts with the marker fixed to the first antibody. After the second incubation step, the antibody complex is washed again to remove unreacted colour or enzyme tagged antibody. Either the intensity of the colour is determined, or in the case of an enzyme tagged antibody, the antibody is exposed to a substrate which is converted by the enzyme to produce an end product. The amount of colour or enzyme tagged antibody in contact with the marker is proportional to the amount of marker present in the body fluid sample. The concentration of the end product, and hence the amount of marker, is preferably determined by a spectrophotometer which measures the optical absorption of light by the end product. This readout is then compared against a standard value for both antigen negative and antigen positive samples.

U.S. Pat. No. 4,517,288 relates to a method for conducting a solid phase enzyme immunoassay of a fluid sample. Accordingly, one method of detection according to the present invention involves an inert porous medium wherein a binding material is immunologically immobilized and includes the steps of immunologically immobilizing a binding material within a finite zone of an inert porous medium, applying a blood coagulation activity marker comprised in a body fluid sample to the zone containing the immobilized binding material, applying a labeled indicator such as a coloured particle or a flourescent marker to the zone which becomes immobilized within the zone in an amount which can be correlated to the amount of marker in the zone, applying a solvent to substantially the center of the zone to chromatographically separate the unbound labeled indicator from the zone, and measuring the amount of labeled indicator remaining in the zone,

U.S. Pat. No. 5,714,389 pertains to a coloured particle immunoassay. Accordingly, the present invention provides in one embodiment a method for detecting a blood coagulation activity marker in a body fluid sample, said method comprising the steps of;

i) providing a test strip, disposed within a housing, comprising sorbent material and defining a flow path, a sample inlet and, spaced apart from said inlet in said flow path, a test site having immobilized thereon a reporter species comprising a first antibody having a binding site specific for a first epitope of said marker, and a separate control site,

ii) providing a conjugate comprising a colored particulate material coupled to a reporter species comprising a second antibody having a binding site specific for a second epitope of said marker,

iii) applying to said inlet said body fluid sample,

iv) transporting to said test site and said control site by sorption, capillary action, wicking, or wetting along said flow path said body fluid sample in admixture with said conjugate thereby producing,

at said control site, a color indicative of a valid test result and indicative that conjugate has bound specifically or non-specifically at said control site, and

at said test site, a specific binding reaction product comprising said marker and an aggregate of said colored particulate material to produce a visibly detectable colour indicative of the presence of said marker.

U.S. Pat. No. 4,446,232 in one aspect relates to a method for determining the presence of antigens in a biological fluid. Accordingly, the present invention in one embodiment relates to a method for determining the presence of a blood coagulation activity marker in a body fluid sample, said method comprising the steps of:

i) bringing a body fluid sample into contact with a device having a matrix including a first zone containing a) bound and immobilized marker and b) a reporter species comprising an enzyme-linked antibody capable of immunologically reacting with said marker, said antibody being positioned in said first zone, and said antibody being removed from said first zone when reacting with marker passing through said first zone, and said antibody being not removed from said first zone in the absence of marker, and a second zone separated from said first zone and containing a substrate capable of reacting with said enzyme-linked antibody to produce a color forming reaction indicating the presence of said antibody,

ii) allowing said body fluid sample to permeate said device: and

iii) observing the presence or absence of any color change in said second zone to thereby determine the presence or absence of the marker being tested for in said fluid.

U.S. Pat. No. 5,710,005 relates to a method for determining the concentration of an analyte in a sample. Accordingly, the present invention in one aspect relates to a method for determining the concentration of a blood coagulation activity marker in a body fluid sample, said method comprising the steps of

i) providing a body fluid sample comprising a blood coagulation activity marker,

ii) establishing a blood coagulation activity marker gradient in a lateral flow device, by said analyte gradient being established by

a) applying said body fluid sample to a defined sample application region comprising an absorbent material,

b) applying a diluent to a defined diluent application region comprising an absorbent material, and

c) bringing said sample application region into contact with said diluent application region to establish a blood coagulation activity marker gradient front.

iii) contacting said gradient with an indicator zone containing a movable and quantifiably detectable reporter species capable of either a) binding said marker or b) competing with said marker for binding to a non-movable and quantifiably detectable reporter species contained in a test zone,

iv) contacting said indicator zone with said test zone containing said non-movable and quantifiably detectable reporter species, wherein said non-movable and quantifiably detectable reporter species binds to said marker or said movable and quantifiably detectable reporter species, and

v) generating a detectable signal indicating the concentration of said marker in said body fluid sample.

Resorter species

Suitable reporter species preferably comprises at least one targeting species, however reporter species may also comprise more than one targeting species. Preferably, the targeting species is an antibody or a fragment thereof capable of specifically detecting a blood coagulation activity marker according to the invention. The detection of the reporter species including a quantifiable detection preferably occurs by detecting a label or marker operably linked or attached to the targeting species in question. Preferred labels and tags are described herein above and further below.

In one embodiment, the at least one antibody comprises a polyclonal antibody or a fragment thereof. However, it is preferred that the at least one antibody comprises a monoclonal antibody or a fragment thereof specific against a blood coagulation marker.

The reporter species in one embodiment further comprises at least one polypeptide operably linked to said at least one antibody. Operably linked as used herein shall be understood to comprise the terms “linked to”, preferably by means of a chemical bond or otherwise, and “correlatable to”, depending on the circumstances. The polypeptide preferably comprises an enzyme capable of cleaving a substrate into a quantifiably detectable product. The enzyme preferably comprises an enzymatic activity selected from the group consisting of a peroxidase activity, including a horseradish peroxidase activity, a glucose oxidase activity, a glucose peroxidase activity, a galactose oxidase, a galactose peroxidase, a oxidoreductase, a beta-glucuronidase activity, a beta-glucosidase activity, a beta-D-galactosidase activity, a phosphatase activity, including an alkaline phosphatase activity, a catalase activity, and a urease activity.

In another embodiment the reporter species comprises at least one fluorochrome operably linked to said at least one antibody. In yet another embodiment the reporter species comprises at least one radio label operably linked to said at least one antibody.

There is also provided an embodiment wherein the reporter species comprises two antibodies, preferably selected from the group consisting of a polyclonal antibody and a monoclonal antibody.

In a further embodiment, at least one quantifiably detectable reporter species according to the invention, such as, but not limited to, at least one antibody comprising a tag, label or marker, further comprises a water-soluble polymeric carrier molecule. The at least one quantifiably detectable reporter species is preferably attached to said polymeric carrier molecule by means of a covalent bond mediated by a reactive group, preferably, but not limited to, a reactive group comprising divinyl sulfone, or a derivative thereof. In the case of divinyl sulfone, the attachment of each of the reactive groups to the polymeric carrier molecule is formed via a covalent bond formed between one of the two vinyl groups of a divinyl sulfone molecule and a reactive functionality on the carrier molecule. The is attachment of the reporter species to the reactive group is thus formed via another covalent bond formed between the other vinyl group originating from the divinyl sulfone molecule and a reactive group present on the reporter species.

Accordingly, in one embodiment of the invention, at least one quantifiably detectable reporter species further comprises a water-soluble polymeric carrier molecule having covalently attached thereto one or more moieties capable of acting as a reactive group which, when activated, is capable of forming a covalent bond between the polymeric carrier molecule and the reporter species. The reactive group preferably comprises groups selected from divinyl sulfone, 4-fluoro-3-nitrophenyl azide, acyl azides such as benzoyl azide and p-methylbenzoyl azide, azido formates such as ethyl azidoformate, phenyl azidoformate, sulfonyl azides such as benzenesulfonyl azide, phosphoryl azides such as diphenyl phosphoryl azide and diethyl phosphoryl azide, diazo compounds such as diazoacetophenone and 1-trifluoromethyl-1-diazo-2-pentanone, diazoacetates such as t-butyl diazoacetate and phenyl diazoacetate, beta-keto-alpha-diazoacetates such as t-butyl alpha diazoacetoacetate, aliphatic azo compounds such as azobisisobutyronitrile, diazirines such as 3-trifluoromethyl-3-phenyldiazirine, ketenes (—CH═C═O) such as ketene and diphenylketene, photoactivatable ketones such as benzophenone and acetophenone, peroxy compounds such as di-t-butyl peroxide, dicyclohexyl peroxide, diacyl peroxides such as dibenzoyl peroxide and diacetyl peroxide, and peroxyesters such as ethyl peroxybenzoate.

Apart from photoactivation, the activation of the reactive group may also take place by e.g. irradiation including gamma irradiation and UV irradiation.

A sulfone group including a divinyl sulfone is a preferred activatable reactive group forming the moiety connecting the carrier molecule and the reporter species. In the case of e.g. divinyl sulfone, each of the moieties is attached via a linkage formed between one of the two vinyl groups of a divinyl sulfone molecule and a reactive functionality on the polymeric carrier molecule, and at least one such moiety in its attached state has the remaining vinyl group free and capable of reaction with the reporter species having a functional group which is reactive towards the free vinyl group.

It is understood that the term “reporter species” in the context of the present invention comprises species such as e.g. any molecule or ionic species capable of serving as a label or marker. Preferred labels and markers are enzymes, fluorescent or luminescent species, and molecules capable of acting as targetting species, i.e. molecules which are capable of binding selectively or specifically to one or more target molecules, moieties, receptors or epitopes, such as e.g. haptens, hapten conjugates, antigens, antibodies, nucleotide sequences, hormones and the like.

Owing to the connection between the polymeric carrier molecule and the reporter species, the establishment, on the polymeric carrier molecule, of covalently bound reactive moieties deriving from e.g. divinyl sulfone, and the establishment of covalent bonds between, on the one hand, such moieties, and, on the other hand, reporter species as defined herein, the known pattern of reactivity of the vinyl groups in a species such as divinyl sulfone will generally require that the reactive functionality on the polymeric carrier, i.e. the group with which a vinyl group of divinyl sulfone will react to form a covalent bond, is a nucleophilic function

Polymeric carrier molecules according to the invention preferably has reactive groups such as e.g.:

—O⁻ (e.g. deprotonated phenolic hydroxy groups, such as deprotonated aromatic hydroxy groups in tyrosine residues of polypeptides or proteins),

—S⁻ (e.g. deprotonated thiol groups on aromatic rings or aliphatic groups, such as deprotonated thiol groups in cysteine residues of polypeptides or proteins),

—OH (e.g. aliphatic hydroxy groups on sugar rings, such as glucose or other monosaccharide rings in oligo- or polysaccharides; or alcoholic hydroxy groups in polyols, such as polyethylene glycols; or hydroxy groups in certain amino acid residues of polypeptides or proteins, such as serine or threonine residues),

—SH (e.g. thiol groups in cysteine residues of polypeptides or proteins), primary amino groups (e.g. in lysine or ornithine residues of polypeptides or proteins: or in amino-substituted sugar rings in certain polysaccharides or derivatives thereof, such as chitosan) or secondary amino groups (e.g. in histidine residues of polypeptides or proteins).

Additionally preferred functional groups are e.g. a N-hydroxysuccinimide group, an aldehyde group, an isocyanate group, an epoxide group, or a sulfphone group,

For similar reasons, the reactive group in question on reporter species in the context of the invention also preferably comprise a nucleophilic action, such as a nucleophilic action mediated by any one of the above-described types The water-soluble polymers capable of acting as carrier molecules carrying the reporter species according to the invention are chosen from a wide variety of types of polymers; including:

natural and synthetic polysaccharides including plant cell wall polysaccharides and bacterial polysaccharides, as well as derivatives thereof, for example dextrans and derivatives thereof, starches and starch derivatives, cellulose and derivatives thereof, glycogen, chitin, xylan, mannan, arabinan, galactan, alginate, laminarin, agar, carrageenan, peptidoglycan, teichoic acid, lipopolysaccharides, xanthan, curdlan, amylose, amylopectin and pectin, including any derivative thereof, as well as certain natural gums and derivatives thereof, such as gum arable and salts of alginic acid;

homopoly(amino acid)s having suitable reactive functionalities, such as polylysines, polyhistidines or polyornithines;

natural and synthetic polypeptides and proteins, such as bovine albumin and other mammalian albumins; and

synthetic polymers having nucleophilic functional groups, such as polyvinyl alcohols, polyallyl alcohol, polyethylene glycols and substituted polyacrylates.

Particularly preferred polymers for the purposes of the present invention are polysaccharides and derivatives thereof, for example; dextrans, carboxy-methyl-dextrans, hydroxyethyl- and hydroxypropyl-starches, glycogen, agarose derivatives, and hydroxyethyl- and hydroxypropyl-celluloses.

Dextran and derivatives thereof represents one presently most preferred polymeric carrier molecule.

In one embodiment, the reporter species and/or the polymeric carrier molecule do not have a net charge, since the presence of a net positive or negative charge may lead, inter alia, to an undesirable, non-specific binding of the reporter species and/or the polymeric carrier molecule to substances and/or materials other than those of interest In many cases, this condition will, unless charged reporter species are introduced, be fulfilled simply by ensuring that the polymeric carrier itself possesses no net charge.

In a further aspect of the invention, the polymeric carrier molecule is, in its free state, substantially linear and substantially uncharged at a pH in the range of from about 4 to about 10, such as from about pH=4 to pH=7, for example from pH=7 to about pH=10, preferably including any pH interval of practical relevance for the vast majority of immunochemical procedures, hybridization procedures and other applications of the reporter species according to the invention. Among the various polymers meeting this criterions are, for example, numerous polysaccharides and polysaccharide derivatives, e.g. dextrans and hydroxyethyl- and hydroxypropylcelluloses.

The water-soluble polymeric carrier preferably has a peak molecular weight ranging from about 1,000 to about 40,000,000. Peak molecular weights of interest are in the range of from about 1,000 to about 80,000, and in the range of from about 80,000 to about 2,000,000. A peak molecular weight of particular interest, notably in the case of dextrans as polymeric carriers, is a peak molecular weight of about 500,000.

The term “peak molecular weight” (also denoted “peak average molecular weight”) as employed herein denotes the molecular weight of greatest abundance, i.e. the molecular weight (among a distribution of molecular weights) which is possessed by the greatest number of molecules in a given sample or batch of the polymer. A manufacturer or distributor will be able to provide reliable peak molecular weight data (determined, for example, by gel-permeation chromatography) which can provide a basis for the selection of a suitable polymer fraction. Peak molecular weight values cited herein refer to the peak molecular weight of the free polymer in question. Cross-linked polymer units will, on average, have higher molecular weights than the individual free polymer molecules from which they are formed.

A further embodiment of the invention relates to reporter species comprising a polymeric carrier molecule having

i) a peak molecular weight of about 500,000 or about 2,000,000, or a peak molecular weight in any one of the following ranges, from about 1,000 to about 20,000; from about 20,000 to about 80,000; from about 80,000 to about 500,000; from about 500,000 to about 5,000,000; or from about 5,000,000 to about 40,000,000, and

ii) having a content of a free, reactive group according to the invention, preferably, but not limited to, a reactive vinyl group, said content of said free, reactive group being either in the range of from about 1 to about 5,000 μmoles of free, reactive groups per gram of polymeric carrier molecule, or in any of the following sub-ranges, expressed in μmoles of reactive groups per gram of polymeric carrier molecule: From about 1 to about 50; from about 50 to about 300; from about 300 to about 1,000; or from about 1,000 to about 5,000.

A quantifiably detectable reporter species according to the present invention, including any reporter species comprising a polymeric carrier molecule according to the invention, preferably further comprises at least one quantifiably detectable tag, marker or label, such as a tag, marker or label selected from the group consisting of a protein, such as ferritin, phycoerythrin, phycocyanin or phycobilin; an enzyme, including peroxidase enzyme, including horseradish peroxidase enzyme, glucose oxidase enzyme, glucose peroxidase enzyme, galactose oxidase enzyme, galactose peroxidase enzyme, oxidoreductase enzyme, beta-glucuronidase enzyme, beta-glucosidase enzyme, beta-D-galactosidase enzyme, phosphatase enzyme, including alkaline phosphatase enzyme, catalase enzyme, and urease enzyme; a toxin, a drug; a dye; a fluorochrome including any fluorescent compound, a luminescent compound, a phosphorescent compound including any other light-emitting substance; a metal-chelating substance, such as iminodiacetic acid, ethylenediaminetetraacetic acid (EDTA), diethylenetriaminepentaacetic acid (DTPA), and desferroxamine B; a substance labelled With a radioactive isotope; and a substance labelled with a heavy atom.

Additional prefered quantifiably detectable reporter species are those comprising a tag, label or marker, either alone or in combination with any one or more of the above mentioned tags, labels or markers, selected from the group consisting of fluorescent substances including fluorescein, preferably fluorescein isothiocyanate (FITC), fluoresceinamine, 1-naphthol, 2-naphthol, eosin, erythrosin, morin, o-phenylenediamine, rhodamine and 8- anilino-1-naphthalenesulfonic acid. Radioactive isotopes of relevance may be selected, for example, among isotopes of hydrogen (i.e. tritium, .sup3 H), carbon (such as .sup.14 C), phosphorus (such as .sup.32 P), sulfur (such as .sup.35 S), iodine (such as sup131 I), bismuth (such as .sup.212 Bi), yttrium (such as .sup.90 Y), technetium (such as sup.99m Tc), palladium (such as .sup.109 Pd) and samarium (such as .sup.153 Sm).

Heavy atoms of relevance may be selected, for example, among Mn, Fe, Co, Ni, Cu, Zn, Ga, In, Ag, Au, Hg, I, Bi, Y, La, Ce, Eu and Gd. Gold (Au), optionally in combination with silver (Ag) as an enhancement reagent

Further preferred reporter species are species capable of selectively binding to, or selectively reacting with, a complementary molecule or a complementary structural region of a blood coagulation activity marker. Examples of such reporter species are, for example, antigens; haptens; monoclonal or polyclonal antibodies; gene probes; natural or synthetic oligo- or polynucleotides; natural or synthetic mono-, oligo- or polysaccharides; lectins; avidin or streptavidin; biotin; growth factors; hormones; receptor molecules; protein A and protein G.

Examples of preferred antibodies according to the invention are sheep anti-human pro-thrombin (Cedarlane, CL 20110A)₁ sheep anti-human pro-thrombin (F.II) (Cedarlane, CL 20111OAP), sheep anti-human pro-thrombin fragment 1 (Cedarlane, CL 20111AP), sheep anti-human pro-thrombin fragment 2 (Cedarlane, CL 20112AP), rabbit anti-human pro-thrombin (Dako, No. A 0325), sheep anti-human pro-thrombin fragment 1 (Affinity Biologicals SAFII-F1AP), monoclonal anti-human pro-thrombin (Biodesign, N77100M), and polyclonal antibody to pro-thrombin (Biogenesis, No. 7880-0004).

Further preferred antibodies useful for detecting a blood coagulation activity marker, preferably a marker comprising a fragment of pro-thrombin according to the present invention, are reported by Hursting et al. (Clin. Chem., 1993, vol. 39(4), p. 583-591), who raised monoclonal antobodies against fragment 1+2 of pro-thrombin based on an amino acid sequence reported by Degen et al. (1983), and Pelzer et al. (Thromb. Haemostas., 1991, vol. 65, p. 153-159), who raised monoclonal antobodies against fragment 1+2 of pro-thrombin based on an amino acid sequence reported by Walz et al. (1983).

Further useful antibodies for detecting a blood coagulation activity marker comprising a fragment of pro-thrombin according to the present invention, are reported by Boisclair et al. C(Thrombosis and Haemostasis, 1993, vol. 70(2), p. 253-258), Bezeaud and Guillin (British J. Haemotology, 1984, vol. 58, p. 597-606), and Rosenberg et al. (J. Biol. Chem., 1979, vol. 254, p. 8751-8761).

In one particularly preferred embodiment there is provided a reporter species comprising a monoclonal antibody and binding fragments thereof that specifically bind to an epitope on the carboxy terminus of a pro-thrombin activation peptide. The epitope preferably comprises the amino acid sequence -Ser-Asp-Arg-Ala-Ile-Glu-Gly-Arg-OH, and the monoclonal antibody is preferably secreted by the hybridoma deposited with the ATCC under Accession No HB 10291, as disclosed in U.S. Pat. No. 5,830,681.

The invention also pertains to reporter species comprising at least one antibody, or a binding fragment thereof, capable of detecting at least one blood coagulation marker defined by the monoclonal antibody deposited with the ATCC under Accession No. HB 10291. The term “defined by” shall be understood to mean that said at least one blood coagulation marker is detected by or reacts with said monoclonal antibody deposited with the ATCC under Accession No. HB 10291.

In one embodiment of the invention, the reporter species comprises an antibody, preferably a monoclonal antibody, against pro-thrombin fragment F₁₊₂ including any functional variant or binding fragment thereof, capable of detecting said blood coagulation marker.

There is also provided a reporter species comprising at least one antibody, or a binding fragment thereof, capable of detecting at least one blood coagulation marker defined by an antibody, preferably a monoclonal antibody, against pro-thrombin fragment F₁.

Also provided is a reporter species comprising at least one antibody, or a binding fragment thereof, capable of detecting at least one blood coagulation marker defined by an antibody, preferably a monoclonal antibody, against pro-thrombin fragment F₂.

In a further embodiment there is provided a reporter species comprising at least one antibody, or a binding fragment thereof, capable of detecting at least one blood coagulation marker defined by an antibody, preferably a monoclonal antibody, against FpA, including any functional variant or binding fragment thereof.

Also provided is a reporter species that comprises an antibody against FpA, or a binding fragment thereof, capable of detecting said blood coagulation marker.

The invention also pertains to a reporter species that comprises at least one antibody, or a binding fragment thereof, capable of detecting at least one blood coagulation marker defined by an antibody against Xₐ.

There is also provided a reporter species comprising an antibody, preferably a monoclonal antibody, against Xₐ, or a binding fragment thereof, capable of detecting said blood coagulation marker.

The blood coagulation activity marker is preferably detectable according to the present invention in an amount of less than 3 nmol/L, such as less than 2.5 nmol/L, for example less than 2.0 nmol/L, such as less than 1.5 nmol/L, for example less than 1.0 nmol/L, such as less than 0.8 nmol/L, for example less than 0.6 nmol/L, such as less than 0.5 nmol/L, for example less than 0.4 nmol/L, such as less than 0.3 nmol/L, for example less than 0.2 nmol/L, such as less than 0.1 nmol/L, for example less than 0.05 nmol/L, such as less than 0.03 nmol/L, for example less than 0.01 nmol/L.

A reporter species according to one embodiment preferably has a molecular weight of at least about 2,000, and in another embodiment the reporter species has a molecular weight of about 2,000 at the most.

The polymeric carrier molecule preferably has from 1 to about 10,000 reporter species covalently attached thereto, for example from about 10 to about 1000 reporter species, such as from about 20 to about 500 reporter species covalently attached thereto. In the latter case, i.e. for reporter species of molecular weight about 2,000 or above, the polymeric carrier molecule of the conjugate may have from 1 to about 1000 reporter species covalently attached thereto, for example from 1 to about 500 reporter species, such as from 1 to about 100, from 2 to about 50, or from about 10 to about 50 reporter species covalently attached thereto.

In another embodiment of the invention, the reporter species comprises at least two antibodies, both of which are preferably attached to a polymeric carrier molecule according to the invention. Accordingly, there is provided at least one quantifiably detectable first reporter species and at least one quantifiably detectable second reporter species are attached to said water-soluble polymeric carrier molecule, wherein each of said first and second reporter species are different and each attached to said polymeric carrier molecule via a reactive group, preferably, but not limited to, a reactive group derived from divinyl sulfone.

Said first and second reporter species are preferably selected from the group of antibodies consisting a pro-thrombin fragment F₁₊₂ antibody, or a binding fragment thereof, a pro-thrombin fragment F₁ antibody, or a binding fragment thereof, a pro-thrombin fragment F₂ antibody, or a binding fragment thereof, a fibrinogen peptide A (FpA) antibody, or a binding fragment thereof, and an X, antibody, or a binding fragment thereof.

Kit of parts

In one embodiment the invention provides a kit of parts suitable for detecting a blood coagulation marker in a body fluid sample The kit of parts comprises an assay device for assaying a body fluid sample for the presence and/or concentration of a blood coagulation activity marker.

The invention further provides a system for detecting a blood coagulation marker in a body fluid sample. Said system may be any suitable assay system and/or a kit of parts system. Examples of the system are discussed above, in particular in relation to the dipstick model and the microfluid device model. However the invention is not limited to these model system, and may be described more generically as follows.

The assay device preferably comprises:

i) a zone for applying a body fluid sample comprising a blood coagulation activity marker, said zone comprising at least one movable reporter species capable of binding said marker, said application zone being in liquid contact with

ii) a zone for detecting the presence, amount or concentration of said at least one reporter species bound to said marker, said zone further comprising a binding species for immobilizing onto said detection zone at least a substantial amount of said marker comprised in said body fluid sample, and optionally

iii) a positive control zone generating a positive control confirming the transfer of at least part of said body fluid sample from said application zone to said detection zone.

The part of the body fluid sample to be assessed may be applied to the zone for applying the sample by any suitable manner, such as dripping the body fluid onto the zone, or arranging the zone directly into a squirt of the body fluid, such as a urine squirt. In another embodiment the body fluid sample may be applied to the zone by dipping the device into the body fluid. A dipstick is normally used in the latter manner, but may of course also be used for applying the body fluid sample in drops onto the zone without dipping the device into the body fluid.

The at least one reporter species comprised in the sample application area preferably comprises an antibody comprising at least one tag, linker or marker that makes it possible at least to detect the presence of said marker, and preferably also makes it possible to quantifiably detect said antibody and/or said reporter species bound to said marker.

The binding species of the detection zone is preferably also an antibody, but this antibody may not comprise any tag, label or marker. It is thus possible to immobilise onto the detection zone an amount of a quantifiably detectable reporter species that accurately reflects the amount of marker present in the body fluid sample. The at least one tag, label or marker used preferably allows both visual detection, by means of the generation of e.g. electromagnetic radiation or a visible colour, and quantification of e.g. the emitted electromagnetic radiation.

Movable reporter species shall be understood to comprise a reporter species capable of moving on e.g. a solid or semi-solid surface, e.g. when being applied to a lateral flow device.

In one embodiment of this aspect of the invention there is provided an assay device for detecting a blood coagulation activity marker present in a body fluid sample, said device comprising:

i) a hollow casing having a body fluid sample application aperture and a test result observation aperture,

ii) a bibulous body fluid sample receiving member within said hollow casing to receive said body fluid sample applied to said sample application aperture,

iii) a test strip comprising a dry porous carrier such as nitrocellulose within said casing and extending from said bibulous body fluid sample receiving member to and beyond said test result observation aperture, said dry porous carrier having a test result zone observable through said observation aperture,

iv) at least one of said bibulous body fluid sample receiving member and said test strip containing upstream from said test result zone a detectable reporter species capable of specifically binding said marker to form a first complex,

v) said reporter species comprising at least one particulate label, such as a dye sol, a metallic sol or a coloured latex particle, and optionally also at least one fluorescently detectable label, said label being released into a mobile form by said body fluid sample,

wherein mobility of said label within said test strip is facilitated by either coating at least a portion of said test strip upstream from said test result zone with a material comprising a polysaccharide, or drying said label onto a portion of said test strip upstream from said test zone in the presence of a material comprising a polysaccharide, in an amount effective to reduce interaction between said test strip and said label, and

wherein said dry porous carrier contains in said test result zone a means for binding said first complex, said means for binding comprising specific binding means immobilized in said test result zone, and

wherein migration of said body fluid sample from said bibulous sample receiving member into and through said dry porous carrier conveying by capillarity said first complex to said test result zone of said dry porous carrier whereat said binding means binds said first complex thereby to form a second complex, and

vi) determining the presence, amount or concentration of said second complex being observable through said test result observation aperture.

In another embodiment there is provided an assay device for detecting a blood coagulation activity marker in a body fluid sample, said device comprising a solid support including at least one detectable reporter species on a test area of the solid support, said at least one detectable reporter species being capable of binding said marker, said reporter species further comprising a liposome or a microcapsule comprising a visible particulate dye compound and optionally also a fluorescently detectable marker.

In yet another embodiment there is provided an assay device comprising

i) a sample application area comprising a predetermined amount of a reporter species comprising an antibody capable of binding said marker deposited thereon, said area being in fluid communication with

ii) a reaction zone comprising a mobilizable reporter species comprising an antibody capable of binding said marker, said reporter species further comprising at least one visually detectable particle and/or at least one fluorescently detectable particle, and

iii) a detection zone comprising a reporter species comprising an antibody capable of binding said marker,

wherein, when said body fluid sample comprising said marker is applied to said sample application area, a threshold amount of the marker is bound to said antibody and thereby prevented from binding to the antibody being present in the reaction zone, and

wherein the marker remaining unbound in said body fluid sample passes from the sample application area through said reaction zone, where it is bound to said mobilizable reporter species comprising i) an antibody capable of binding said marker, and ii) at least one visually detectable particle and/or at least one fluorescently detectable particle, and

wherein the marker bound to the mobilizable reporter species is brought into contact with the detection zone, where the marker is bound to said reporter species comprising said antibody capable of binding said marker, and

wherein said binding of said marker results in immobilization of said mobilizable reporter species further comprising i) an antibody capable of binding said marker, and ii) at least one visually detectable particle and/or at least one fluorescently detectable particle,

Body fluid samples

The body fluid sample is preferably a sample excreted from the body whereby the sample may be obtained without invasive techniques, such as a urine sample, a saliva sample, or a sample comprising body perspiration.

However, the body fluid sample may also be from body fluid normally obtained by invasive techniques, such as blood samples, which include whole blood samples, fractionated blood samples, including plasma samples, and samples comprising one or more of erythrocytes, leukocytes and thrombocytes.

Biological species correlatable to the blood coagulation activity

Biological species correlatable with the blood coagulation activity are preferably selected from the group consisting of pro-thrombin, thrombin, thrombin anti-thrombin III complex (TAT), fibrinogen, fibrin, fibrin/fibrinogen degradation products such as FDP D-dimer, and alpha 2PI plasmin complex (PIC).

Clinical conditions

A clinical condition within the meaning of said term as applied herein pertains to any clinical condition influencing the coagulation system. Accordingly, the present invention may be used for monitoring the coagulation system in relation to a wide variety of disorders and/or diseases.

Patients to be subjected to a surgical treatment are often routinely administered anti-coagulants, such as heparin, either as a combined pre- and post-surgical treatment or as a post-surgical treatment alone. Today the patients are routinely offered the anti-coagulant treatment due to the lack of reliable, fast and simple monitoring methods, and the treatment regime is often several weeks after the surgical treatment. Accordingly, in one embodiment the method of the present invention is used for monitoring patients having been subjected to a surgical treatment. Thereby, the patients themselves or the medical staff may, preferably non-invasively, and on a daily basis, monitor the coagulation status of the patient to diagnose the patients in need of treatment. Thereby all patients not suffering from a coagulation disturbance post-surgically will not be subjected to the anti-coagulant treatment, and furthermore, the patients in need thereof may be administered the most appropriate dosage of anticoagulants, instead of the routinely administered dosages.

The monitoring may be carried out by the medical staff during hospitalisation, but in many embodiments of the invention the monitoring may be carried out by the patients themselves, for example as home management.

The result of the monitoring may either be registered directly by the patient or the medical staff conducting the monitoring test and reported to the physician responsible for the treatment However, it is envisaged by the present invention that the result of the monitoring is directly reported to the responsible physicial or clinic by means of computer and telecommunication technique, whereby the treatment can be initiated If necessary without any delay

Such monitoring routines are also applicable in all other situations wherein the coagulation system is to be monitored, such situations arising e.g. for a patient suffering from:

any cardiac disease, such as angina, or myocardial infarction, or patients in anti-thrombotic treatment after heart surgery,

any vasculatory disorder or disease, such as venous thrombosis, arterial thrombosis, and transitory cerebral,

any renal diseases, such as nephrotic syndrom,

any inherent or acquired coagulation disorders, such as Protein S deficiency, protein C deficiency, Antithrombin III deficiency, homocysteinaemia, factor V Leiden, genemutation, and Lupus anticoagulant,

any hepatic diseases, such as liver cirrhosis,

any kind of inflammatory diseases having an impact on the coagulation system, such as Bowel inflammatory diseases, and rheumatoid arthritis,

any hormone disorders, such as diabetes, and

during the progress of infections, in particular infections that may lead to septicaemia, in which situations the risk of disseminated intra-vascular coagulation may arise.

The methods of the present invention may be applied initially and during the progress of many injuries often leading to a transient coagulation disorder, for example due to tissue damage and bleeding. In such situations the monitoring of the coagulation system may be an indication of the severeness of the injury.

Furthermore, pregnancy may lead to a coagulation disorder, in particular in relation to pre-eclampsia.

Also, the methods may be applied during routine control of patients being administered oral contraceptives, oestrogen therapy, and other treatments having an impact on the coagulation system.

Furthermore, patients being treated with an anti-coagulant medicament may be monitored regularly, such as daily or weekly, in order for medically qualified personel to react quickly to any change in the coagulation system that should desirably result in a change in the administration of an anti-coagulant medicament.

A disorder of the coagulation system may be the first clinical sign of a disease or disorder, such as e.g. a cancer that has not given rise to any other symptoms yet.

Accordingly, the methods of the invention may be used for diagnosing a coagulation disorder that may be caused by a disease not yet diagnosed. In such a situation, diagnosis of the coagulation disorder may optionally result in the application of other diagnostic methods in order to more specifically diagnose the disease or disorder in question.

Today, other such indicators of diseases are used routinely, such as the measurement of sedimentation, of protein C₁ and other non-specific markers of disease. By the present invention a new non-invasive marker may be applied routinely in the primary diagnosis of diseases and disorders.

Monitoring or diagnosing methods according to the invention may be carded out by performing initially the method according to the invention on a body fluid sample.

The body fluid sample may be any sample obtainable by non-invasive methods, such as a urine sample, a saliva sample, a perspiration sample. It is preferred to use a urine sample. The urine sample may be a sample obtained during urination, if however the patient has been supplied with a catheter the urine sample may be obtained through the catheter as well. Preferably the urine sample is obtained as the morning urination.

A few drops of urine is then applied to the assay as described above, and after a suitable reaction time the result is registered.

### EXAMPLES

### Example 1

F1+2 levels in plasma and urine in healthy volunteers and patients undergoing total hip or knee replacement surgery

The present study was undertaken to evaluate the level of F1+2 in plasma and urine in healthy volunteers, and to evaluate the levels of F1+2 in plasma and urine in patients undergoing total hip or knee replacement surgery in relation to type and time of operation, Furthermore, the study was undertaken to determine the correlation between F1+2 in plasma and urine. The study was a single centre, prospective, cohort study.

Materials and methods

Healthy Volunteers

5 healthy individuals were willing to participate in this study

Inclusion Criteria

Primary osteoarthrosis of hip or knee

Primary hip or knee prosthesis

Or revision of either

Exclusion Criteria

Denied informed consent

Age <18 years

Ethnics

The study was approved by the local ethics committee and all patients gave informed written consent before inclusion.

Patients

It was decided to study cemented and uncemented procedures and to include a total of 18 patients. 6 patients undergoing cemented THR, 6 undergoing uncemented THR and 6 undergoing uncemented TKR with an equal representation of women and men.

Surgical Treatment

All operations used standard procedures, surgical exposures and standard implants. Anaesthesia was spinal/or epidural. Postoperative treatment was standard for this kind of surgery with early mobilization and weight bearing as soon as possible. Thromboprophylaxis was administered to all patients with Clexane (enoxaparin) 40 mg o.d. s.c., and started 12 h before the operation and continued for at least 7 days. Physical therapy was used from the first postoperative day until discharge from hospital using a standard program The clinical course of each patient was followed until day 35 after the operation.

Blood Sampling

Samples were taken preoperatively day -1 (on the day before surgery) and postoperatively day 1-6 (day of operation is day 1), on the day of discharge and on day 35 between 8 and 9 a.m. Each sample consisted of 20 ml citrated whole blood that was immediately centrifuged and the plasma was snap frozen and stored at −80 ° C. until analysis.

Urine Sampling

24 h urine specimens were collected on day -1-day 7, on the day of discharge and on day 35. In addition spot urine samples were obtained every morning on the same days. The samples were stored at −80° C. until analysis.

Laboratory Tests

For all analyses of F1+2 in either plasma or urine a commercially available Kit was used: Enzygnost F1+2 ELISA kit from Dade Behring Marburg GMBH, D-35041 Marburg, Germany and performed according to the manufacturers instructions. Reference interval (5^{th}-95^{th} percentile): 0.44-1.11 nmol/L.

For plasma concentration for 10-fold determination in one assay at two levels the cv (deviation coefficient) was 10,42% for 3,12 mmol/L and 11,03% for 0,80 nmol/L. For urine pool the cv was 10,96% for 0,07 nmol/L. Lower limit of measurement for plasma and urine was 0,04 nmol/L.

Clinical Registrations

Sex, age, height, weight and surgical data were registered on all included patients. Surgical data comprised of date of operation, duration of operation, type of prosthesis (cemented/uncemented), complications, day of mobilisation. The entire clinical course for each patient including day of discharge.

Results

5 healthy volunteers (3 men and 2 women) participated in the study 12 patients (9 men and 9 women) had a THR (6 cemented and 6 uncemented) and 6 had a TKR (uncemented). No surgical complications were registered during the study? All patients had a normal serum creatinine during the study

Conclusions

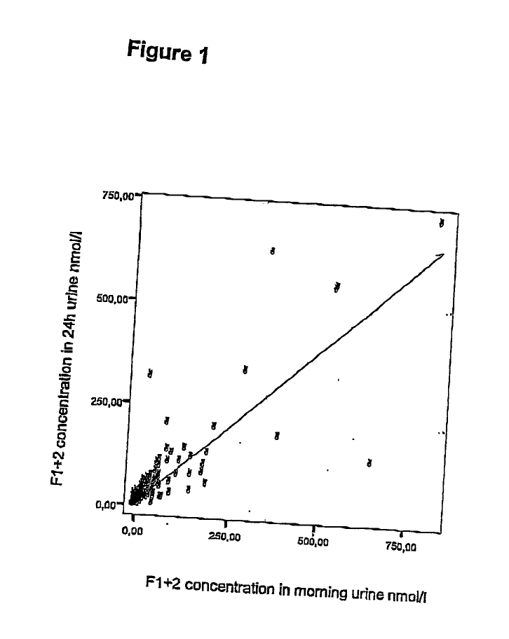

The study clearly shows that F1+2 is detectable in urine and that spot measurement (morning urine) highly correlates with 24 h urine sampling. FIG. 1 illustrates the correlation between the concentration in nmol/l of Fragment 1+2 in 24 h urine and morning urine samples The linear regression shows that

F1+2-conc. in 24 h urine (mmol/l)=7.69+0.78×F1+2-conc. in morning urine

R-Square=0.71

This implicates that urine concentration measurement can be done in the morning by a single sampling

There is a significant correlation between plasma level and urine concentration of F1+2 measured over time in 18 patients. 5 controls showed that normal values of plasma concentration of F1+2 results in <0.05 nmol/L Based on these results we have selected a cut off level for Actiwatch of >0.3 nmol/L to indicate that a patient is in a hypercoagulate state.

TABLE 1

| | | F1 + 2- | F1 + 2- | | | | | | |
|---|---|---|---|---|---|---|---|---|---|
| | | conc. in | conc. in | F1 + 2- | | | | | Prim- |
| | | 24 h | morning | conc. in | TAT-conc. | | | | ary/ |
| | | urine | urine | plasma | in plasma | cem/ | | hip/ | revis- |
| | Day | [nmol/l] | [nmol/l] | [nmol/l] | [ug/l] | ucem | sex | knee | ion |
| U1 | | | | | | U | M | H | p |
| | D-1 | 0.09 | 0.06 | 1.88 | 6.3 | | | | |
| 23/6-24/6 | OP = D 1 | 0.17 | 0.13 | 0.77 | 2.7 | | | | |
| | D 2 | 0.32 | 0.27 | 1.01 | 2.2 | | | | |
| | D 3 | 8.40 | | 1.69 | 5.1 | | | | |
| | | | — | | | | | | |
| | D 4 | 12.9 | 6.59 | 1.93 | 4.2 | | | | |
| | D 5 | 5.54 | 5.36 | 1.89 | 3.6 | | | | |
| | D 6 | 7.26 | 8.28 | 2.20 | 4.2 | | | | |
| | D 7 | | 9.59 | 2.16 | 5.8 | | | | |
| | | — | | | | | | | |
| | Discharge | | | | | | | | |
| | End = D40 | 0.26 | 0.18 | 1.31 | 5.1 | | | | |
| U2 | | | | | | U | M | h | p |
| | D-1 | <0.04 | <0.04 | 0.56 | <2.0 | | | | |
| 23/6-24/6 | OP = D 1 | 0.06 | 0.07 | 0.66 | <2.0 | | | | |
| | D 2 | 0.13 | 0.16 | 0.97 | 2.3 | | | | |
| | D 3 | 0.09 | 0.13 | 1.41 | 5.1 | | | | |
| | D 4 | 0.11 | 0.09 | 1.92 | 4.9 | | | | |
| | D 5 | 0.08 | 0.07 | 2.02 | 4.7 | | | | |
| | D 6 | 0.07 | 0.10 | 1.79 | 4.4 | | | | |
| | D 7 | 0.05 | 0.04 | 1.60 | 4.1 | | | | |
| | Discharge = D 8 | <0.04 | <0.04 | 1.37 | 3.3 | | | | |
| | End = D 35 | <0.04 | <0.04 | 2.33 | 16.6 | | | | |
| U3 | | | | | | C | F | h | p |
| | D-1 | 0.05 | 0.11 | 1.55 | 2.6 | | | | |
| 22/7-23/7 | OP = D 1 | 0.52 | 0.38 | 2.36 | 18.7 | | | | |
| | D 2 | 0.56 | 0.99 | 1.60 | 14.0 | | | | |
| | D 3 | 0.66 | 0.67 | 4.09 | 21.9 | | | | |
| | D 4 | 0.62 | 0.42 | 3.89 | 21.8 | | | | |
| | D 5 | 0.42 | 0.25 | 3.09 | 11.8 | | | | |
| | D 6 | 0.23 | 0.06 | 2.48 | 8.4 | | | | |
| | D 7 | 0.10 | 0.14 | 2.07 | 6.7 | | | | |
| | Discharge = D 15 | 0.05 | 0.04 | 4.89 | 49.1 | | | | |
| | End = D 36 | 0.09 | 0.11 | 2.59 | 2.1 | | | | |
| U4 | | | | | | U | F | k | p |
| | D-1 | 0.13 | 0.12 | 1.30 | 2.1 | | | | |
| 30/8-31/8 | OP = D 1 | 0.05 | 0.54 | 2.34 | 27.0 | | | | |
| | D 2 | 0.79 | 1.15 | 1.63 | 13.3 | | | | |
| | D 3 | <0.04 | 0.07 | 2.01 | 10.1 | | | | |
| | D 4 | <0.04 | 0.16 | 2.11 | 10.1 | | | | |
| | D 5 | 0.22 | 0.31 | 1.79 | 6.9 | | | | |
| | D 6 | 0.30 | 0.50 | 1.90 | 5.7 | | | | |
| | D 7 | 0.70 | | 2.29 | 8.0 | | | | |
| | | | — | | | | | | |
| | Discharge = D 14 | 0.08 | 0.25 | 2.38 | 9.7 | | | | |
| | End = D 35 | 0.13 | 0.21 | 2.00 | 6.3 | | | | |
| U5 | | | | | | u | F | k | p |
| | D-1 | <0.04 | <0.04 | 1.13 | <2.0 | | | | |
| 23/8-24/8 | OP = D 1 | <0.04 | 0.04 | 1.28 | 8.7 | | | | |
| | D 2 | <0.04 | <0.04 | 0.93 | 5.4 | | | | |
| | D 3 | <0.04 | 0.04 | 1.12 | 4.9 | | | | |
| | D 4 | <0.04 | <0.04 | 1.34 | 2.4 | | | | |
| | D 5 | <0.04 | <0.04 | 1.46 | 2.3 | | | | |
| | D 6 | <0.04 | 0.04 | 1.53 | 2.2 | | | | |
| | D 7 | <0.04 | <0.04 | 1.85 | 3.5 | | | | |
| | Discharge = D 18 | <0.04 | <0.04 | 0.97 | <2.0 | | | | |
| | End = D 35 | 0.06 | 0.05 | 0.86 | <2.0 | | | | |
| U6 | | | | | | u | m | k | p |
| | D-1 | 0.17 | 0.22 | 1.02 | <2.0 | | | | |
| 30/8-31/8 | OP = D 1 | 2.70 | | 4.15 | 28.3 | | | | |
| | | | — | | | | | | |
| | D 2 | 0.31 | 0.28 | 2.29 | 12.7 | | | | |
| | D 3 | 0.23 | 0.46 | 2.62 | 15.2 | | | | |
| | D 4 | 0.42 | 0.37 | 3.02 | 11.6 | | | | |
| | D 5 | 0.41 | 0.26 | 3.43 | 10.1 | | | | |
| | D 6 | 0.24 | 0.20 | 3.68 | 8.0 | | | | |
| | D 7 | 0.35 | 0.28 | 3.77 | 8.3 | | | | |
| | Discharge = D 8 | 0.21 | 0.20 | 3.87 | 6.9 | | | | |
| | End = D 35 | 0.22 | 0.29 | 2.44 | 10.1 | | | | |
| U7 | | | | | | u | F | h | p |
| | D-1 | 0.20 | 0.17 | 1.66 | <2.0 | | | | |
| 8/9-9/9 | OP = D 1 | 0.82 | 1.53 | 2.60 | 21.8 | | | | |
| | D 2 | 1.06 | 1.89 | 4.66 | 28.0 | | | | |
| | D 3 | 1.97 | 2.13 | 5.56 | 22.9 | | | | |
| | D 4 | 1.10 | 0.70 | 2.30 | 7.1 | | | | |
| | D 5 | 0.37 | 0.42 | 2.72 | 7.4 | | | | |
| | D 6 | 0.37 | 0.26 | 2.89 | 6.9 | | | | |
| | D 7 | 0.45 | 0.24 | 2.90 | 6.6 | | | | |
| | Discharge = D 12 | 0.26 | 0.05 | 1.98 | 4.5 | | | | |
| | End = D 36 | 0.06 | 0.06 | 2.37 | 6.5 | | | | |
| U8 | | | | | | u | F | h | p |
| | D-1 | 0.04 | 0.04 | 0.59 | <2.0 | | | | |
| 8/9-9/9 | OP = D 1 | 0.30 | 0.25 | 1.01 | 7.5 | | | | |
| | D 2 | 0.30 | 0.57 | 1.16 | 7.0 | | | | |
| | D 3 | 0.50 | 0.70 | 1.26 | 4.9 | | | | |
| | D 4 | 0.63 | 0.62 | 1.29 | 3.2 | | | | |
| | D 5 | 1.84 | 3.91 | 1.60 | 3.6 | | | | |
| | D 6 | 1.34 | 1.98 | 1.45 | 2.5 | | | | |
| | D 7 | 0.14 | 0.18 | 1.07 | 2.0 | | | | |
| | Discharge = D 12 | 0.04 | 0.04 | 0.88 | <2.0 | | | | |
| | End = D 36 | 0.06 | 0.07 | 1.07 | <2.0 | | | | |
| U9 | | | | | | u | m | k | r |
| | D-1 | 0.18 | 0.40 | 1.73 | 10.2 | | | | |
| 13/9-14/9 | OP = D 1 | 1.02 | 0.65 | 1.92 | 10.8 | | | | |
| | D 2 | 0.60 | 0.30 | 2.31 | 11.0 | | | | |
| | D 3 | 0.17 | 0.24 | 2.35 | 10.1 | | | | |
| | D 4 | 0.14 | 0.24 | 2.56 | 9.7 | | | | |
| | D 5 | 0.22 | 0.37 | 2.56 | 9.6 | | | | |
| | D 6 | 0.27 | 0.37 | 2.98 | 9.7 | | | | |
| | D 7 | 0.21 | 0.59 | 2.35 | 9.8 | | | | |
| | Discharge = D 11 | 0.22 | 0.33 | 2.93 | 9.4 | | | | |
| | End = D 35 | 0.17 | 0.18 | 2.10 | 9.1 | | | | |
| U10 | | | | | | u | F | h | r |
| | D-1 | 0.23 | | 2.10 | 6.2 | | | | |
| | | | — | | | | | | |
| 16/9-17/9 | OP = D 1 | | 0.60 | 1.83 | 6.1 | | | | |
| | | — | | | | | | | |
| | D 2 | 0.74 | 0.48 | 2.52 | 7.0 | | | | |
| | D 3 | 0.25 | 0.24 | 3.44 | 8.8 | | | | |
| | D 4 | 0.28 | 0.36 | 3.34 | 8.4 | | | | |
| | D 5 | 0.39 | 0.54 | 4.08 | 10.3 | | | | |
| | D 6 | 0.75 | 0.69 | 5.39 | 6.4 | | | | |
| | D 7 | 0.67 | 0.71 | 4.60 | 7.0 | | | | |
| | Discharge = D 13 | 0.50 | 0.45 | 3.00 | 8.0 | | | | |
| | End = D 41 | 0.20 | 0.18 | 2.82 | 17.4 | | | | |
| U12 | | | | | | u | m | h | r |
| | D-1 | 1.11 | 1.24 | 1.31 | 7.2 | | | | |
| 22/9-23/9 | OP = D 1 | 6.33 | 3.51 | 1.59 | 11.7 | | | | |
| | D 2 | 3.39 | 2.93 | 1.68 | 8.6 | | | | |
| | D 3 | 2.03 | 0.85 | 1.91 | 7.6 | | | | |
| | D 4 | 1.09 | 0.91 | 2.32 | 25.2 | | | | |
| | D 5 | 1.39 | 0.88 | 2.27 | 6.3 | | | | |
| | D 6 | 1.42 | 1.35 | 2.50 | 7.2 | | | | |
| | D 7 | 1.29 | 1.02 | 1.87 | 6.3 | | | | |
| | Discharge = D 14 | 0.84 | 0.61 | 1.58 | 3.9 | | | | |
| | End = D 35 | 1.22 | 1.54 | 2.92 | 11.1 | | | | |
| U14B | | | | | | u | m | k | p |
| | D-1 | 0.06 | <0.04 | 1.12 | 5.5 | | | | |
| 4/10-5/10 | OP = D 1 | 0.25 | 0.21 | 1.73 | 33.1 | | | | |
| | D 2 | 0.18 | 0.16 | 1.10 | 19.0 | | | | |
| | D 3 | 0.17 | 0.16 | 1.31 | 14.7 | | | | |
| | D 4 | 0.11 | 0.08 | 1.77 | 13.1 | | | | |
| | D 5 | 0.12 | 0.08 | 1.70 | 9.8 | | | | |
| | D 6 | 0.10 | 0.06 | 1.65 | 7.9 | | | | |
| | D 7 | 0.06 | 0.05 | 1.90 | 7.3 | | | | |
| | Discahrge = D 11 | 0.04 | 0.04 | 1.86 | 8.3 | | | | |
| | End = D 35 | <0.04 | <0.04 | 1.53 | 6.4 | | | | |
| U15 | | | | | | c | m | h | p |
| | D-1 | 0.05 | <0.04 | 1.00 | <2.0 | | | | |
| 23/9-24/9 | OP = D 1 | 0.18 | 0.17 | 1.18 | 4.7 | | | | |
| | D 2 | 0.22 | 0.77 | 0.98 | 5.7 | | | | |
| | D 3 | 0.60 | 1.96 | 1.42 | 6.4 | | | | |
| | D 4 | 0.86 | 1.84 | 1.97 | 5.9 | | | | |
| | D 5 | 0.47 | 0.73 | 2.75 | 6.0 | | | | |
| | D 6 | 0.22 | 0.78 | 2.33 | 6.1 | | | | |
| | D 7 | 0.27 | 0.29 | 2.13 | 3.9 | | | | |
| | Discharge = D 11 | 0.09 | 0.07 | 1.81 | 4.3 | | | | |
| | End = D 35 | 0.04 | <0.04 | 1.59 | <2.0 | | | | |
| U16 | | | | | | c | m | h | p |
| | D-1 | 0.04 | 0.06 | 0.66 | <2.0 | | | | |
| 29/9-30/9 | OP = D 1 | 3.13 | 0.33 | 1.46 | 12.5 | | | | |
| | D 2 | 0.16 | 0.06 | 1.52 | 11.3 | | | | |
| | D 3 | 0.08 | 0.09 | 1.33 | 8.0 | | | | |
| | D 4 | 0.11 | 0.06 | 1.32 | 5.0 | | | | |
| | D 5 | 0.17 | 0.13 | 1.55 | 4.5 | | | | |
| | D 6 | 0.10 | 0.10 | 1.43 | 2.7 | | | | |
| | D 7 | 0.12 | 0.11 | 1.76 | 6.1 | | | | |
| | Discharge = D | | | | | | | | |
| | | — | — | — | — | | | | |
| | End = D | | | | | | | | |
| | | — | — | — | — | | | | |
| U17 | | | | | | c | F | h | p |
| | D-1 | 0.10 | 0.07 | 1.34 | <2.0 | | | | |
| 30/9-1/10 | OP = D 1 | 5.58 | 5.40 | 2.52 | 21.1 | | | | |
| | D 2 | 0.64 | 0.39 | 2.10 | 8.5 | | | | |
| | D 3 | 0.59 | 0.62 | 2.73 | 7.0 | | | | |
| | D 4 | 0.65 | 0.46 | 3.25 | 8.0 | | | | |
| | D 5 | 0.54 | 0.43 | 3.37 | 5.9 | | | | |
| | D 6 | 0.56 | 0.57 | 2.81 | 5.0 | | | | |
| | D 7 | 0.43 | 0.35 | 3.36 | 6.1 | | | | |
| | Discharge = D 13 | 0.20 | 0.19 | 2.83 | 4.0 | | | | |
| | End = D 35 | 0.13 | 0.10 | 2.34 | <2.0 | | | | |
| U18 | | | | | | u | F | k | r |
| | D-1 | 0.20 | 0.22 | 1.59 | 2.2 | | | | |
| 11/10-12/10 | OP = D 1 | 0.23 | 0.26 | 1.41 | 2.6 | | | | |
| | D 2 | 0.32 | 1.00 | 1.16 | 2.7 | | | | |
| | D 3 | 0.40 | 1.52 | 1.37 | 2.5 | | | | |
| | D 4 | 0.35 | 0.21 | 1.73 | 2.6 | | | | |
| | D 5 | 0.18 | 0.22 | 2.13 | 21.8 | | | | |
| | D 6 | 0.17 | 0.30 | 2.11 | 2.3 | | | | |
| | D 7 | 0.23 | 0.45 | 2.19 | 2.4 | | | | |
| | Discharge = D 11 | 0.19 | 0.05 | 2.03 | 2.6 | | | | |
| | End = D 35 | 0.15 | 0.08 | 2.27 | 3.6 | | | | |
| U20 | | | | | | c | m | h | p |
| | D-1 | 0.09 | 0.09 | 1.25 | <2.0 | | | | |
| 15/10-16/10 | OP = D 1 | 0.97 | 0.71 | 1.65 | 7.1 | | | | |
| | D 2 | 0.52 | 0.33 | 1.35 | 5.6 | | | | |
| | D 3 | 0.33 | 0.11 | 1.79 | 4.7 | | | | |
| | D 4 | 0.23 | 0.12 | 2.06 | 3.9 | | | | |
| | D 5 | 0.25 | 0.18 | 2.04 | 3.9 | | | | |
| | D 6 | 0.25 | 0.13 | 1.99 | 3.2 | | | | |
| | D 7 | 0.27 | 0.15 | 2.05 | 2.0 | | | | |
| | Discharge = D | | | | | | | | |
| | | — | — | — | — | | | | |
| | End = D 35 | 0.06 | 0.04 | 1.91 | 2.6 | | | | |
| U21 | | | | | | c | F | h | p |
| | D-1 | <0.04 | <0.04 | 1.37 | <2.0 | | | | |
| 20/10-21/10 | OP = D 1 | | 0.08 | 1.44 | 14.1 | | | | |
| | | — | | | | | | | |
| | D 2 | 0.06 | 0.05 | 1.26 | 9.0 | | | | |
| | D 3 | | 0.05 | 2.22 | 9.5 | | | | |
| | | — | | | | | | | |
| | D 4 | 0.08 | 0.11 | 2.07 | 9.9 | | | | |
| | D 5 | 0.09 | 0.04 | 2.43 | 8.1 | | | | |
| | D 6 | 0.09 | 0.04 | 2.06 | 5.9 | | | | |
| | D 7 | 0.05 | 0.06 | 2.84 | 4.6 | | | | |
| | Discharge = D | | | | | | | | |
| | | — | — | — | — | | | | |
| | End = D | | | | | | | | |
| | | — | — | — | — | | | | |

| | | F1 + 2- | F1 + 2- | | | |
|---|---|---|---|---|---|---|
| | | konc. i | konc. i | F1 + 2- | | |
| | | døgnurl | morgenu | konc. i | TAT-konc. | |
| | | n | rin | plasma | i plasma | |
| | Dato | [nmol/l] | [nmol/l] | [nmol/l] | [ug/l] | |
| UK1 | 17/11-18/11 | <0.04 | 0.04 | 0.73 | <2.0 | m |
| | 18/11-19/11 | <0.04 | 0.04 | 0.66 | <2.0 | |
| UK2 | 16/11-17/11 | 0.04 | 0.05 | 1.01 | 5.2 | F |
| | 17/11-18/11 | 0.05 | 0.04 | 1.37 | 4.6 | |
| UK3 | 16/11-17/11 | <0.04 | <0.04 | 0.59 | <2.0 | m |
| | 17/11-18/11 | <0.04 | <0.04 | 0.52 | <2.0 | |
| UK4 | 16/11-17/11 | <0.04 | 0.04 | 1.14 | <2.0 | m |
| | 17/11-18/11 | 0.05 | <0.04 | 1.42 | <2.0 | |
| UK5 | 16/11-17/11 | <0.04 | <0.04 | 0.97 | <2.0 | k |
| | 17/11-18/11 | <0.04 | <0.04 | 1.00 | <2.0 | |

Table 1. F1+2 in blood and urine samples and TAT levels in blood samples. TAT is considered to be an indicator of ongoing activation of the blood coagulation system, Following abbreviations are used: U uncemented type of prosthesis, C cemented type of prothesis, M male, F female, H hip, K knee, P primary, R revision.

TABLE 2

| | F1 + 2-conc. in | F1 + 2-conc. in | |
|---|---|---|---|
| | 24 h urine | morning urine | F1 + 2-conc. in |
| Day | [nmol/l] | [nmol/l] | plasma [nmol/l] |
| D-1 | 0.09 | 0.06 | 1.88 |
| OP = D 1 | 0.17 | 0.13 | 0.77 |
| D 2 | 0.32 | 0.27 | 1.01 |
| D 4 | 12.9 | 6.59 | 1.93 |
| D 5 | 5.54 | 5.36 | 1.89 |
| D 6 | 7.26 | 8.28 | 2.20 |
| End = D 40 | 0.26 | 0.18 | 1.31 |
| D-1 | 0.03 | 0.03 | 0.56 |
| OP = D 1 | 0.06 | 0.07 | 0.66 |
| D 2 | 0.13 | 0.16 | 0.97 |
| D 3 | 0.03 | 0.13 | 1.41 |
| D 4 | 0.11 | 0.09 | 1.92 |
| D 5 | 0.08 | 0.07 | 2.02 |
| D 6 | 0.07 | 0.10 | 1.79 |
| D 7 | 0.00 | 0.04 | 1.60 |
| Discharge = D 8 | 0.03 | 0.03 | 1.37 |
| End = D 35 | 0.03 | 0.03 | 2.33 |
| D-1 | 0.05 | 0.11 | 1.55 |
| OP = D 1 | 0.52 | 0.38 | 2.36 |
| D 2 | 0.68 | 0.99 | 1.60 |
| D 3 | 0.88 | 0.87 | 4.09 |
| D 4 | 0.62 | 0.42 | 3.89 |
| D 5 | 0.42 | 0.25 | 3.09 |
| D 6 | 0.23 | 0.06 | 2.48 |
| D 7 | 0.10 | 0.14 | 2.07 |
| Discharge = D 15 | 0.05 | 0.04 | 4.89 |
| End = D 36 | 0.09 | 0.11 | 2.59 |
| D-1 | 0.13 | 0.12 | 1.30 |
| OP = D 1 | 0.05 | 0.54 | 2.34 |
| D 2 | 0.79 | 1.15 | 1.63 |
| D 3 | 0.03 | 0.07 | 2.01 |
| D 4 | 0.03 | 0.16 | 2.11 |
| D 5 | 0.22 | 0.31 | 1.79 |
| D 6 | 0.30 | 0.50 | 1.90 |
| Discharge = D 14 | 0.08 | 0.25 | 2.38 |
| End = D 35 | 0.13 | 0.21 | 2.00 |
| D-1 | 0.03 | 0.03 | 1.13 |
| OP = D 1 | 0.03 | 0.04 | 1.28 |
| D 2 | 0.03 | 0.03 | 0.93 |
| D 4 | 0.03 | 0.03 | 1.34 |
| D 5 | 0.03 | 0.03 | 1.46 |
| D 6 | 0.03 | 0.04 | 1.53 |
| D 7 | 0.03 | 0.03 | 1.85 |
| Discharge = D 18 | 0.03 | 0.03 | 0.97 |
| End = D 35 | 0.06 | 0.05 | 0.86 |
| D-1 | 0.17 | 0.22 | 1.02 |
| D 2 | 0.31 | 0.28 | 2.29 |
| D 3 | 0.23 | 0.46 | 2.62 |
| D 4 | 0.42 | 0.37 | 3.02 |
| D 5 | 0.41 | 0.26 | 3.43 |
| D 6 | 0.24 | 0.20 | 3.68 |
| D 7 | 0.35 | 0.28 | 3.77 |
| Discharge = D 12 | 0.21 | 0.20 | 3.87 |
| End = D 35 | 0.22 | 0.29 | 2.44 |
| D-1 | 0.20 | 0.17 | 1.66 |
| OP = D 1 | 0.82 | 1.53 | 2.60 |
| D 2 | 1.06 | 1.89 | 4.66 |
| D 3 | 1.97 | 2.13 | 5.56 |
| D 4 | 1.10 | 0.70 | 2.30 |
| D 5 | 0.37 | 0.42 | 2.72 |
| D 6 | 0.37 | 0.26 | 2.89 |
| D 7 | 0.45 | 0.24 | 2.90 |
| Discharge = D 12 | 0.26 | 0.05 | 1.98 |
| End = D 36 | 0.06 | 0.06 | 2.37 |
| D-1 | 0.04 | 0.04 | 0.59 |
| OP = D 1 | 0.30 | 0.25 | 1.01 |
| D 2 | 0.30 | 0.57 | 1.16 |
| D 3 | 0.50 | 0.70 | 1.26 |
| D 4 | 0.63 | 0.62 | 1.29 |
| D 5 | 1.84 | 3.91 | 1.60 |
| D 6 | 1.34 | 1.98 | 1.45 |
| D 7 | 0.14 | 0.18 | 1.07 |
| Discharge = D 12 | 0.04 | 0.04 | 0.83 |
| End = D 36 | 0.06 | 0.07 | 1.07 |
| D-1 | 0.18 | 0.40 | 1.73 |
| OP = D 1 | 1.02 | 0.65 | 1.92 |
| D 2 | 0.60 | 0.30 | 2.31 |
| D 3 | 0.17 | 0.24 | 2.35 |
| D 4 | 0.14 | 0.24 | 2.56 |
| D 5 | 0.22 | 0.37 | 2.56 |
| D 6 | 0.27 | 0.37 | 2.98 |
| D 7 | 0.21 | 0.59 | 2.35 |
| Discharge = D 11 | 0.22 | 0.33 | 2.93 |
| End = D 35 | 0.17 | 0.18 | 2.10 |
| D 2 | 0.74 | 0.48 | 2.52 |
| D 3 | 0.25 | 0.24 | 3.44 |
| D 4 | 0.28 | 0.35 | 3.34 |
| D 5 | 0.39 | 0.54 | 4.06 |
| D 6 | 0.75 | 0.69 | 5.39 |
| D 7 | 0.67 | 0.71 | 4.60 |
| Discharge = D 13 | 0.50 | 0.45 | 3.00 |
| End = D 41 | 0.20 | 0.18 | 2.82 |
| D-1 | 1.11 | 1.24 | 1.31 |
| OP = D 1 | 6.33 | 3.51 | 1.59 |
| D 2 | 3.38 | 2.93 | 1.68 |
| D 3 | 2.03 | 0.85 | 1.91 |
| D 4 | 1.09 | 0.91 | 2.32 |
| D 5 | 1.39 | 0.88 | 2.27 |
| D 6 | 1.42 | 1.35 | 2.50 |
| D 7 | 1.29 | 1.02 | 1.87 |
| Discharge = D 14 | 0.84 | 0.61 | 1.58 |
| End = D 35 | 1.22 | 1.54 | 2.92 |
| D-1 | 0.06 | 0.03 | 1.12 |
| OP = D 1 | 0.25 | 0.21 | 1.73 |
| D 2 | 0.18 | 0.16 | 1.10 |
| D 3 | 0.17 | 0.16 | 1.31 |
| D 4 | 0.11 | 0.08 | 1.77 |
| D 5 | 0.12 | 0.08 | 1.70 |
| D 6 | 0.10 | 0.06 | 1.65 |
| D 7 | 0.06 | 0.05 | 1.90 |
| Discharge = D 11 | 0.04 | 0.04 | 1.86 |
| End = D 35 | 0.03 | 0.03 | 1.53 |
| D-1 | 0.05 | 0.03 | 1.00 |
| OP = D 1 | 0.18 | 0.17 | 1.18 |
| D 2 | 0.22 | 0.77 | 0.98 |
| D 3 | 0.60 | 1.96 | 1.42 |
| D 4 | 0.86 | 1.84 | 1.97 |
| D 5 | 0.47 | 0.73 | 2.75 |
| D 6 | 0.22 | 0.78 | 2.33 |
| D 7 | 0.27 | 0.29 | 2.13 |
| Discharge = D 11 | 0.09 | 0.07 | 1.81 |
| End = D 35 | 0.04 | 0.03 | 1.59 |
| D-1 | 0.04 | 0.05 | 0.66 |
| OP = D 1 | 3.13 | 0.33 | 1.46 |
| D 2 | 0.18 | 0.06 | 1.52 |
| D 3 | 0.08 | 0.08 | 1.33 |
| D 4 | 0.11 | 0.06 | 1.32 |
| D 5 | 0.17 | 0.13 | 1.55 |
| D 6 | 0.10 | 0.10 | 1.43 |
| D 7 | 0.12 | 0.11 | 1.76 |
| D-1 | 0.10 | 0.07 | 1.34 |
| OP = D 1 | 5.58 | 5.40 | 2.52 |
| D 2 | 0.64 | 0.39 | 2.10 |
| D 3 | 0.59 | 0.62 | 2.73 |
| D 4 | 0.65 | 0.48 | 3.25 |
| D 5 | 0.54 | 0.43 | 3.37 |
| D 6 | 0.56 | 0.57 | 2.81 |
| D 7 | 0.43 | 0.35 | 3.36 |
| Discharge = D 13 | 0.20 | 0.19 | 2.83 |
| End = D 35 | 0.13 | 0.10 | 2.34 |
| D-1 | 0.20 | 0.22 | 1.59 |
| OP = D 1 | 0.23 | 0.26 | 1.41 |
| D 2 | 0.32 | 1.00 | 1.16 |
| D 3 | 0.40 | 1.52 | 1.37 |
| D 4 | 0.35 | 0.21 | 1.73 |
| D 5 | 0.18 | 0.22 | 2.13 |
| D 6 | 0.17 | 0.30 | 2.11 |
| D 7 | 0.23 | 0.45 | 2.19 |
| Discharge = D 11 | 0.19 | 0.05 | 2.03 |
| End = D 35 | 0.15 | 0.08 | 2.27 |
| D-1 | 0.09 | 0.09 | 1.25 |
| OP = D 1 | 0.97 | 0.71 | 1.65 |
| D 2 | 0.52 | 0.33 | 1.35 |
| D 3 | 0.33 | 0.11 | 1.79 |
| D 4 | 0.23 | 0.12 | 2.06 |
| D 5 | 0.25 | 0.18 | 2.04 |
| D 6 | 0.25 | 0.13 | 1.99 |
| D 7 | 0.27 | 0.15 | 2.05 |
| End = D 35 | 0.06 | 0.04 | 1.91 |
| D-1 | 0.03 | 0.03 | 1.37 |
| D 2 | 0.06 | 0.05 | 1.26 |
| D 4 | 0.08 | 0.11 | 2.07 |
| D 5 | 0.09 | 0.04 | 2.43 |
| D 6 | 0.09 | 0.04 | 2.06 |
| D 7 | 0.05 | 0.06 | 2.84 |
| 17/11-18/11 | 0.03 | 0.04 | 0.73 |
| 16/11-19/11 | 0.03 | 0.04 | 0.66 |
| 16/11-17/11 | 0.04 | 0.05 | 1.01 |
| 17/11-18/11 | 0.05 | 0.04 | 1.37 |
| 16/11-17/11 | 0.03 | 0.03 | 0.59 |
| 17/11-18/11 | 0.03 | 0.03 | 0.52 |
| 16/11-17/11 | 0.03 | 0.04 | 1.14 |
| 17/11-15/11 | 0.05 | 0.03 | 1.42 |
| 16/11-17/11 | 0.03 | 0.03 | 0.97 |
| 17/11-18/11 | 0.03 | 0.03 | 1.00 |

Table 2 F1+2 in blood and urine samples. Table 2 is a selection of table 1, however some of the value are indicated more accurately

TABLE 3
Correlations between F1 + 2 concentration in plasma and morning urine

| | | F1 + 2-conc. | F1 + 2-conc. |
|---|---|---|---|
| | | in plasma | in morning urine |
| | | [nmol/l] | [nmol/l] |
| Spearman's F1 + 2-conc. in | Correlation Coefficient | 1.000 | .438 |
| rho plasma | | | |
| [nmol/l] | | | |
| | Sig. (2-tailed) | | .000 |
| | N | 175 | 175 |
| F1 + 2-conc. in | Correlation Coefficient | .438 | 1.000 |
| morning urine | | | |
| [nmol/l] | | | |
| | | | 000 |
| | Sig. (2-tailed) | .000 | |
| | N | 175 | 175 |

**Correlation is significant at the .01 level (2-tailed).

TABLE 4
Correlations between F1 + 2 concentration in plasma and 24 h urine

| | | F1 + 2-conc. | F1 + 2-conc. |
|---|---|---|---|
| | | in plasma | in 24 h urine |
| | | [nmol/l] | [nmol/l] |
| Spearman's F1 + 2-conc. | Correlation Coefficient | 1.000 | .459 |
| rho in plasma | | | |
| [nmol/l] | | | |
| | Sig. (2-tailed) | | .000 |
| | N | 175 | 175 |
| F1 + 2-conc. | Correlation Coefficient | .459 | 1.000 |
| in 24 h urine | | | |
| [nmol/l] | | | |
| | | | 000 |
| | Sig. (2-tailed) | .000 | |
| | N | 175 | 175 |

**Correlation is significant at the .01 level (2-tailed).

TABLE 5
Correlation of F1 + 2 concentration in 24 h urine and morning urine

| | | | F1 + 2-conc. in morning |
|---|---|---|---|
| | | F1 + 2-conc. in | urine |
| | | 24 h urine [nmol/l] | [nmol/l] |
| Spearman's rho F1 + 2-conc. in | Correlation Coefficient | 1.000 | .907 |
| 24 h urine | | | |
| [nmol/l] | | | |
| | Sig. (2-tailed) | | .000 |
| | N | 175 | 175 |
| F1 + 2-conc. in | Correlation Coefficient | .907 | 1.000 |
| morning urine | | | |
| [nmol/l] | | | |
| | | | 000 |
| | Sig. (2-tailed) | .000 | |
| | N | 175 | 175 |

**Correlation is significant at the .01 level (2-tailed).

### Example 2

Dipstick for measuring prothrombin F1+2 in a bodyfluid sample

A dipstick for measuring prothrombin F1+2 in a bodyfluid sample that could clearly distinguish between a concentration of prothrombin F1+2 in said bodyfluid sample above and below a given cut-off point, by the appearance of a clear visually detectable signal, such as a red spot in a functional lateral flow assay was developed.

The antigen to be tested is Prothrombin Fragment 1+2 (Mw 36.000) in urine. Moreover, it was expected that levels of free Fragment 1 (Mw 22.000) and Fragment 2 (Mw 14.000) are measurable as well.

As intact prothrombin is not released to the urine, it is possible to use commercial available antibodies against whole prothrombin for detection of Prothrombin Fragment 1+2. Such an antibody has been used in the production of the conjugate, since this type of antibody is readily available in contrast to specific antibodies against the fragments (Fragment 1 and Fragment 2 antibodies).

Two different targeting species was used. One targeting species was coupled to the solid surface on the dipstick, the so-called catching antibody. This antibody recognised Prothrombin Fragment 27 which means that the test will recognise Prothrombin F1+2 as well as free fragment 2. The antibody was a Sheep anti Human Prothrombin Fragment 2, (Affinity Biologicals, Inc.; cat. no: SAFII-F2AP). This fragment 2 specific antibody was chosen, since it gives a better signal and a better cut-off than if a Fragment I specific antibody is used.

The reporter species comprised the second targeting species, which was an antibody recognising whole prothombin, and it was a Rabbit anti-Human Prothrombin antibody, (DAKO AIS; cat.no: A0325).

The reporter species further comprised polydextran polymeric carrier molecules, which were of approximately 500.000 Da, to which the reactive group divinylsulphone were covalently attached. The second targeting species were attached to the polydextran chains via these active groups. Furthermore, the reporter species comprised rhodamine label molecules, which were also attached via the divinylsulphone groups

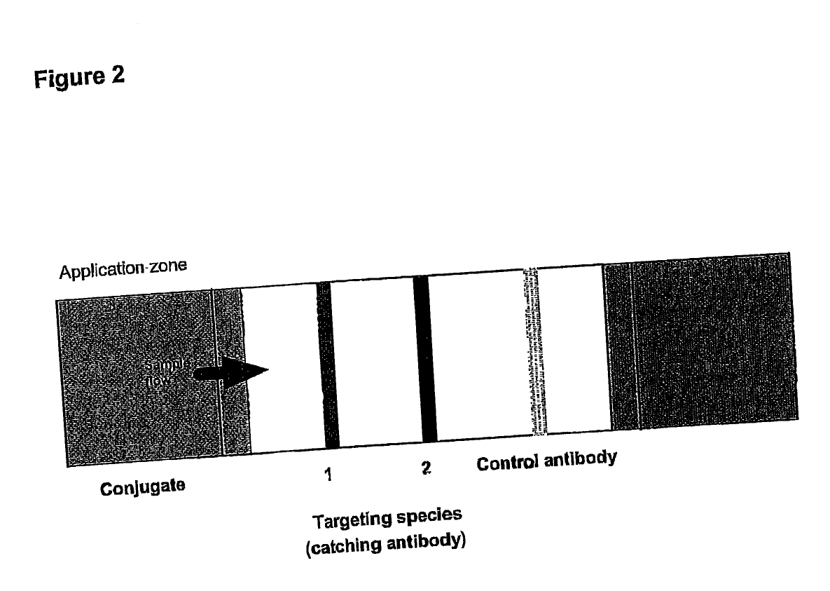

To test the reporter species a 2-layer lateral flow test was employed, following the principles outlined in FIG. 2. FIG. 2 illustrates a schematic dipstick, for use in an assay for testing a blood coagulation activity marker in a body fluid sample. The dipstick comprises an application zone for the sample comprising the reporter species. The term conjugate refers to reporter species. Furthermore, the dipstick comprises one zone whereto the catching antibody is coupled and a second zone whereto the control antibody is coupled. The dipstick is made of nitrocellulose.

A secondary antibody with specificity against the targeting antibody comprised within the reporter species was used as catching antibody. This lateral test gave a positive red spot, which showed that 1) targeting antibody was coupled to polydextran carrier; 2) the polydextran carrier had good flow characteristics conjugate. Furthermore, none of them gave rise to background/unspecific binding.

To test whether the reporter species were functional when applying a real urine test comprising Fragment 1 and 2, a 3-layer lateral flow test was used. For this purpose the catching antibody outlined above (Sheep anti Human Prothrombin Fragment 2,) was used. The reporter species was then eluted with urine, in which the concentration of Fragment 1 and 2 had previously been tested. Urine with about 5 nmol/L F1+2 were used in these tests as positive samples. This test illustrated that it is possible to distinguish clearly between positive and negative urine samples in a lateral flow test. The control spot gave a clear positive signal in all tests. The flow test did not show any unspecific binding. All reporter species showed good flow characteristics on nitrocellulose membrane used in the tests. Two reporter species were especially useful for a dipstick for diagnostic testing and were used in the experiments below.

The levels of prothrombin F1+2 in urine used during the development of the dipstick was pre-determined. The urine samples were derived from patients with an elevated level of Prothrombin F1+2, and from a control group with a Prothrombin F1+2 level <0.04 nmol/L.

The urine samples were stored at 4° C. for three months. Subsequently, the samples were divided into smaller amounts and stored at −20° C. The samples did not show any sign on degradation.

The reference interval used in the test was from 0.04 to 12.9 nmol/L. One preferred cut-off value was 0.3 nmol/L, however different reporter species comprising different polymeric carrier molecules were developed, which gave the opportunity of producing different cut-off values within a certain range, The cut-off values are based on available sample urine. Two examples were made:

Reporter species 1: Cut-off: 0.85 nmol/L

Reporter species 2: Cut-off: 0.13 nmol/L

The test was developed so that a visually visible red spot appears when the test is positive. This spot is produced by accumulation of rhodamine linked to the reporter species. The positive result in the test is defined as samples comprising Prothrombin F1+2 levels higher than the cut-off value is used. A negative result, which is visualised by no colour change (no red spot appear), was obtained when urine samples with Prothrombin F1+2 levels lower than the cut-off value was used.

The test is a 1-step test, where urine is applied directly to the dipstick after which the test results appear. When the test is performed as a 1-step test the first colour reaction appear on the flow test as early as after 1-3 minutes. The test is finished after about 5 minutes.

A control antibody that binds the reporter species independently of the antigen in the urine, was also coupled to the solid surface of the dipstick within the control zone. A red control spot appeared every time in the test regardless whether negative urine or positive urine was used, as an indicator of whether the test was correctly performed. The red colour of this control spot was also produced by accumulation of rhodamine linked to the reporter species.

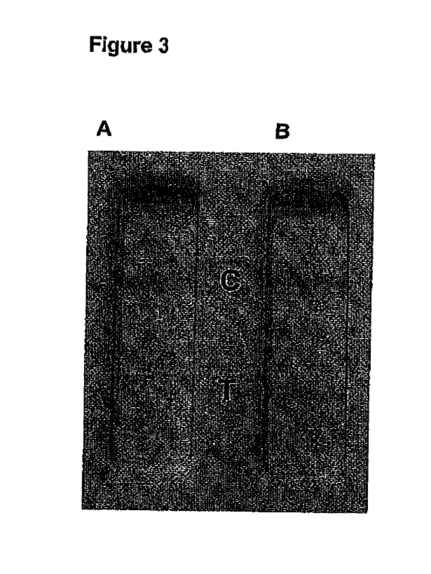

Furthermore, a dipstick has been developed so that a red test line appears across the membrane instead of a red spot, both for observing the test result and the control (FIG. 3). Often R is observed that the colour intensity is increased on a test line compared to a test spot.

During the development of the test no components in urine other than Prothrombin F1+2 has been identified to affect the test results, meaning that no “false positives” have been identified.

The manufacturer of the catching antibody against Prothrombin Fragment 2, informs that the antibody reacts with free Fragment 2, intact prothrombin, and intermediates wherein Fragment 2 is bound(=Prothrombin F1+2). The manufacturer of the antibody against Prothrombin coupled to the reporter species informs that the antibody reacts with intact prothrombin, Gla-deficient prothrombin (Gla domain is in the Fragment 1 region and Gla-deficient is from this point of view defined as Fragment 2). Hence, the test recognizes Prothrombin F1+2, and Prothrombin Fragment 2 in urine.

### Example 3

Competetive dispstick

In this example a dipstick similar to the dipstick described in Example 2 was produced as a competitive dipstick whereby a positive signal is shown as no change of colour, whereas a negative signal is shown as a colour change.

To achieve this, Prothrombin from Human plasma (cat.no; 539515, Calbiochem) was coupled to the solid surface on the dipstick, in stead of the catching antibody. The reporter species was similar to the one used in example 2.

The amount of reporter species was titrated in a way such as a red spot (visible accumulation of rhodamine) only appeared in negative samples

1. Method of correlating a predetermined amount of at least one blood coagulation activity marker comprised in a sample with the amount of at least one quantifiably detectable reporter species capable of being operably linked to said blood coagulation activity marker, said method comprising the steps of
i) obtaining a test sample comprising a predetermined amount of at least one blood coagulation activity marker:
ii) obtaining at least one quantifiably detectable reporter species capable of being operably linked to said blood coagulation activity marker,
iii) contacting said test sample comprising said predetermined amount of at least one blood coagulation activity marker with said at least one quantifiably detectable reporter species,
iv) operably linking said predetermined amount of said blood coagulation activity marker comprised in said test sample to said at least one quantifiably detectable reporter species,
v) detecting said at least one quantifiably detectable reporter species operably linked to said predetermined amount of said blood coagulation activity marker comprised in said test sample,
vi) determining the amount of said at least one quantifiably detectable reporter species operably linked to said predetermined amount of said blood coagulation activity marker comprised in said test sample, and
vii) correlating said predetermined amount of said blood coagulation activity marker comprised in said test sample with said determined amount of said at least one quanffiably detectable reporter species.

2. Method according to claim 1, wherein said sample is contacted with at least one target species prior to step iv).

3. Method of determining the amount of at least one blood coagulation activity marker comprised in a body fluid sample, said method comprising the steps of
i) obtaining a body fluid sample comprising at least one blood coagulation activity marker,
ii) contacting said body fluid sample comprising said blood coagulation activity marker with at least one quantifiably detectable reporter species,
iii) operably linking said blood coagulation activity marker comprised in said body fluid sample to said at least one quantifiably detectable reporter species,
iv) detecting said at least one quantifiably detectable reporter species operably linked to said blood coagulation activity marker comprised in said body fluid sample,
v) determining the amount of said at least one quantifiably detectable reporter species operably linked to said blood coagulation activity marker comprised in said body fluid sample,
vi) correlating the determined amount of said at least one quantifiably detectable reporter species with the amount of said blood coagulation activity marker comprised in said body fluid sample, and based on the correlation of step vi), determining said amount of said blood coagulation activity marker comprised in said body fluid sample.

4. Method according to claim 3, wherein said body fluid sample is contacted with at least one targeting species prior to step iii).

5. Method of claim 3, wherein said correlation in step vi) is performed by using data obtainable by the method of correlating a predetermined amount of at least one blood coagulation activity marker with the amount of at least one quantifiably detectable reporter species as described in claim 1.

6. Method of correlating the blood coagulation activity of a blood sample obtained from an individual with the amount of at least one blood coagulation activity marker comprised in a body fluid sample obtained from said individual, said method comprising the steps of
i) obtaining a blood sample from said individual,
ii) obtaining a body fluid sample comprising at least one blood coagulation activity marker from said individual,
iii) determining the amount of at least one blood coagulation activity marker present in said body fluid sample obtained from said individual, and
iv) correlating said amount of said at least one blood coagulation activity marker present in said body fluid sample obtained from said individual with said blood coagulation activity of said individual,

7. Method of claim 6 wherein the correlation of the blood coagulation activity of said blood sample obtained from an individual with said amount of at least one blood coagulation activity marker comprised in a body fluid sample obtained from said individual is obtainable by correlating the determined blood coagulation time and the determined amount of said at least one blood coagulation activity marker comprised in said body fluid sample.

8. Method of claim 6, wherein said determination in step iii) is obtainable by the method of determining the amount of at least one blood coagulation activity marker as described in claim 3.

9. Method of claim 6, wherein said determination of said amount of said blood coagulation activity marker as described in claim 3 is obtainable by the method of correlating a predetermined amount of at least one blood coagulation activity marker with the amount of at least one quantifiably detectable reporter species as described in claim 1.

10. Method of correlating the blood coagulation activity of a blood sample obtained from an individual with the amount of at least one blood coagulation activity marker comprised in a body fluid sample obtained from said individual, said method comprising the steps of
i) obtaining a blood sample from said individual,
ii) obtaining a body fluid sample comprising at least one blood coagulation activity marker from said individual,
iii) determining the amount of at least one quantifiably detectable biological species present in said blood sample obtained from said individual, said at least one quantifiably detectable biological species being correlatable to said blood coagulation activity in said blood sample obtained from said individual,
iv) determining the amount of at least one blood coagulation activity marker present in said body fluid sample obtained from said individual, said blood coagulation activity marker being correlatable with said at least one quantifiably detectable biological species present in said blood sample obtained from said individual,
v) correlating said amount of said at least one blood coagulation activity marker present in said body fluid sample obtained from said individual with the amount of at least one quantifiably detectable biological species present in said blood sample obtained from said individual,
vi) correlating said amount of said at least quantifiably detectable biological species present in said blood sample obtained from said individual with the blood coagulation activity of said individual, and
vii) based on the correlations of steps v) and vi), correlating said amount of at least one blood coagulation activity marker present in said body fluid sample obtained from said individual with said blood coagulation activity of said individual.

11. Method of claim 10, wherein said determination in step iv) is obtainable by the method of determining the amount of at least one blood coagulation activity marker as described in claim 3.

12. Method of claim 10, wherein said determination of said amount of said blood coagulation activity marker as described in claim 3 is obtainable by the method of correlating a predetermined amount of at least one blood coagulation activity marker with the amount of at least one quantifiably detectable reporter species as described in claim 1.

13. Method of determining the blood coagulation activity of an individual, said method comprising the steps of
i) obtaining a body fluid sample comprising at least one blood coagulation activity marker from said individual,
ii) determining the amount of said at least one blood coagulation activity marker present in said body fluid sample,
iii) correlating said determined amount of said at least one blood coagulation activity marker present in said body fluid sample with said blood coagulation activity of said individual, and
iv) based on the correlation of step iii), determining said blood coagulation activity of said individual.

14. Method of claim 13, wherein said determination in step ii) is obtainable by the method of determining the amount of at least one blood coagulation activity marker as described in claim 3.

15. Method of claim 14, wherein said determination of said amount of said blood coagulation activity marker as described in claim 3 is obtainable by the method of correlating a predetermined amount of at least one blood coagulation activity marker with the amount of at least one quanffiably detectable reporter species as described in claim 1.

16. Method of claim 13, wherein said correlation in step iii) is obtainable by the method of correlating the blood coagulation activity of a blood sample with the amount of at least one blood coagulation activity marker comprised in a body fluid sample as described in claim 6.

17. Method of claim 11, wherein said correlation in step iii) is obtainable by the method of correlating the blood coagulation activity of a blood sample with the amount of at least one blood coagulation activity marker comprised in a body fluid sample as described in claim 10.

18. Method of monitoring the blood coagulation activity of an individual, said method comprising obtaining a plurality of individual determinations of said blood coagulation activity of said individual, wherein each determination of said blood coagulation activity is obtainable by the method of claim 13.

19. Method of monitoring the blood coagulation activity of an individual, said method comprising obtaining a plurality of individual determinations of said blood coagulation activity of said individual, wherein each determination of said blood coagulation activity is obtainable by the method of claim 14.

20. Method of monitoring the blood coagulation activity of an individual, said method comprising obtaining a plurality of individual determinations of said blood coagulation activity of said individual, wherein each determination of said blood coagulation activity is obtainable by the method of claim 16.

21. Method of monitoring a clinical condition in an individual, said clinical condition affecting the blood coagulation activity in said individual, said method comprising the steps of
i) obtaining over a predetermined period of time a plurality of body fluid samples comprising at least one blood coagulation activity marker from said individual,
ii) determining the amounts of said at least one blood coagulation activity marker present in said plurality of body fluid samples,
iii) correlating said determined amounts of said at least one blood coagulation activity marker present in said plurality of body fluid samples obtained over a predetermined period of time with said clinical condition affecting said blood coagulation activity in said individual, and
iv) based on said correlation of step iii), monitoring said clinical condition in said individual.

22. Method of claim 21, wherein said determination of said amounts of said at least one blood coagulation activity marker present in said plurality of body fluid samples obtained over a predetermined period of time is obtainable by the method of claim 3.

23. Method of claim 22, wherein said determination of said amount of said blood coagulation activity marker as described in claim 3 is obtainable by the method of correlating a predetermined amount of at least one blood coagulation activity marker with the amount of at least one quantifiably detectable reporter species as described in claim 1.

24. Method of claim 2, wherein said body fluid sample is a urine sample.

25. Method of determining the amount of at least one blood coagulation activity marker of claim 2, wherein such determination is based on a cut-off point, above which one visible colour indicates the presence of the marker and below which cut-off point another visible colour or no colour change indicates that the marker is present in an amount less than that indicated cut-off point.

26. Method of claim 25 wherein said cut-off point is at least 0.1 nM, for example at least 0.15 nM, such as at least 0.20 nM, for example at least 0.25 nM, such as at least 0.30 nM, for example between 0.1 and 2.0 nM, for example between 0.20 and 1.5 nM, such as between 0.30 and 1.0 nM of said marker.

27. Method of claim 25 wherein said cutoff point is around 0.30 nM of said marker.

28. Method of claim 10, wherein said biological, species is selected from the group consisting of pro-thrombin, thrombin, thrombin antithrombin III complex (TAT), fibrinogen, fibrin, fibrin/fibrinogen degradation products D-dimer (FDP D-dimer), and alpha 2PI plasmin complex (PIC).

29. Method of claim 1, wherein said blood coagulation activity marker is selected from the group consisting of peptides comprising a fragment of pro-thrombin.

30. Method of claim 28, wherein said marker is selected from the group consisting of peptides comprising pro-thrombin Fragment 1+2 (F₁₊₂), peptides comprising pro-thrombin Fragment 1 (F₁), and peptides comprising pro-thrombin Fragment 2 (F₂).

31. Method of claim 28, wherein said marker is selected from peptides comprising pro-thrombin Fragment 1+2 (F₁₊₂)

32. Method of claim 28, wherein said marker is selected from peptides comprising pro-thrombin Fragment 1 (F₁)

33. Method of claim 28, wherein said marker is selected from peptides comprising prothrombin Fragment 2 (F₂).

34. Method of claim 28, wherein said marker is selected from the group consisting of pro-thrombin Fragment 1+2 (F₁₊₂), pro-thrombin Fragment 1 (F₁), and pro-thrombin Fragment 2 (F₂).

35. Method of claim 28, wherein said marker essentially consists of pro-thrombin Fragment 1+2 (F1+2).

36. Method of claim 28, wherein said marker essentially consists of pro-thrombin Fragment 1 (F₁).

37. Method of claim 28, wherein said marker essentially consists of pro-thrombin Fragment 2 (F₂).

38. Method of claim 28, wherein said marker is pro-thrombin Fragment 1+2 (F₊₂) comprising amino acid residues 1 to 271 of pro-thrombin, including any functional variant thereof being at least 95% identical to said sequence, said functional variant being obtained by deletion, insertion or substitution of at least one amino acid.

39. Method of claim 28, wherein said marker is pro-thrombin Fragment 1 (F₁) comprising amino acid residues 1 to 155 of prothrombin, including any functional variant thereof being at least 95% identical to said sequence, said functional variant being obtained by deletion: insertion or substitution of at least one amino acid.

40. Method of claim 28, wherein said marker is pro-thrombin Fragment 2 (F₂) comprising amino acid residues 156 to 271 of pro-thrombin, including any functional variant thereof being at least 95% identical to said sequence, said variant being obtained by deletion, insertion or substitution of at least one amino acid.

41. Method of claim 1, wherein said marker is detectable by a reporter species capable of detecting any of pro-thrombin Fragment 1+2 (F₁₊₂), pro-thrombin Fragment 1 (F₁), and pro-thrombin Fragment 2 (F₂).

42. Method of claim 1, wherein, said blood coagulation activity marker is selected from the group consisting of peptides comprising a fragment of fibrinogen.

43. Method of claim 42, wherein said marker is selected from the group consisting of peptides comprising fibrinopeptide A (FpA).

44. Method of claim 42, wherein said marker essentially consists of fibrinopeptide A (FpA).

45. Method of claim 42, wherein said marker is fibrinopeptide A (FpA).

46. Method of claim 1, wherein said marker is detectable by a reporter species capable of detecting fibrinopeptide A (FpA).

47. Method of claim 1, wherein said marker is selected from the group consisting of peptides comprising the carboxy-terminal 17 amino acid residues of the heavy chain of Factor Xₐ.

48. Method of claim 47, wherein said marker essentially consists of the carboxy-terminal 17 amino acid residues of the heavy chain of Factor Xₐ.

49. Method of claim 47, wherein said marker is the carboxy-terminal 17 residues of the heavy chain of Factor Xₐ.

50. Method claim 1, wherein said reporter species comprises at least one targeting species.

51. Method of claim 1, wherein said reporter species comprises more than one targeting species.

52. Method according to 50, wherein said targeting species comprises at least one antibody, or a binding fragment thereof, capable of detecting at least one blood coagulation marker defined by an antibody against F₂.

53. Method of claim 52, wherein said targeting species comprises the F₁₊₂ antibody deposited with the ATCC under the Accession No. HB 10291, or a binding fragment thereof, capable of detecting said blood coagulation marker.

54. Method according to 50, wherein said targeting species comprises at least one antibody, or a binding fragment thereof, capable of detecting at least one blood coagulation marker defined by an antibody against F₁.

55. Method of claim 54, wherein said targeting species comprises the antibody against F₁ disclosed by Bezeaud and Guillin in British Journal of Haematology (1984), vol. 58, pages 597-606, or a binding fragment thereof, capable of detecting said blood coagulation marker.

56. Method according to 50, wherein said reporter species comprises at least one antibody, or a binding fragment thereof, capable of detecting at least one blood coagulation marker defined by an antibody against F₂.

57. Method of claim 56, wherein said targeting species comprises the antibody against F₂ deposited with the ATCC under the Accession No. HB 10291, or a binding fragment thereof, capable of detecting said blood coagulation marker.

58. Method according to 50, wherein said, targeting species comprises at least one antibody capable of detecting at least one blood coagulation marker defined by an antibody against FpA.

59. Method of claim 58, wherein said targeting species comprises a binding fragment of said FpA antibody capable of detecting said blood coagulation marker.

60. Method according to claim 50, wherein said targeting species comprises at least one antibody capable of detecting at least one blood coagulation marker defined by an antibody against Xₐ.

61. Method of claim 60, wherein said targeting species comprises a binding fragment of said Xₐ antibody capable of detecting said blood coagulation marker.

62. Method according to claim 52, wherein the targeting species is immobilised on a solid surface.

63. Method according to claim 62, wherein said solid surface is comprised within a lateral flow device.

64. Method according to claim 62, wherein said solid surface is a dipstick or part thereof.

65. Method according to claim 62, wherein said solid surface is nitrocellulose.

66. Method according to claim 62, wherein said solid surface is comprised within a micro fluid device.

67. Method of claim 41, wherein said at least one antibody comprises a polyclonal antibody.

68. Method of claim 41, wherein said at least one antibody comprises a monoclonal antibody.

69. Method according to claim 50, wherein said reporter species further comprises at least one polypeptide operably linked to said at least one targeting species.

70. Method of claim 69, wherein said polypeptide comprises an enzyme

71. Method of claim 70, wherein said enzyme comprises a peroxidase activity.

72. Method according to claim 50, wherein said reporter species further comprises at least one fluorochrome.

73. Method according to claim 50, wherein said reporter species further comprises at least one radio label.

74. Method according claim 50, wherein said reporter species further comprises at least one coloured dye molecule.

75. Method according to claim 74, wherein said at least one coloured dye molecule is rhodamine.

76. Method according to claim 50, wherein said reporter species comprises two antibodies.

77. Method according to claim 50, wherein said boo antibodies are selected from a polyclonal antibody and a monoclonal antibody.

78. Method according to claim 50, wherein said reporter species comprises a polymeric carrier molecule.

79. Method according to 78, wherein the polymeric carrier molecule has a hydrophilic sugar chain backbone.

80. Method according to 78, wherein the polymeric carrier molecule has a polymeric dextran backbone.

81. Method of treatment of a clinical condition in a human or animal body by therapy or surgery, said method comprising the steps of
i) determining the amount of at least one blood coagulation activity marker comprised in a body fluid sample according to the method of claim 6,
ii) correlating said amount of said blood coagulation activity marker in said body fluid sample determined in step i) to said clinical condition,
iii) confirming said correlation of said blood coagulation activity marker in said body fluid sample determined in step 1) to said clinical condition by diagnosing said clinical condition,
iv) based on the diagnosis of step iii) treating said clinical condition in said human or animal body.

82. Method of treatment of a clinical condition in a human or animal body by therapy or surgery, said method comprising the steps of
i) determining the blood coagulation activity of an individual according to the method of claim 13,
ii) correlating said blood coagulation activity of said individual determined in step i) to said clinical condition,
iii) confirming said correlation of said blood coagulation activity of said individual determined in step i) to said clinical condition by diagnosing said clinical condition,
iv) based on the diagnosis of step iii), treating said clinical condition in said human or animal body.

83. Diagnostic method practised on the human or animal body, said method comprising the steps of
i) determining the amount of at least one blood coagulation activity marker comprised in a body fluid sample according to the method of claim 6,
ii) correlating said amount of said blood coagulation activity marker in said body fluid sample determined in step i) to said clinical condition,
iii) based on the correlation of step ii), diagnosing said clinical condition in said human or animal body.

84. Diagnostic method practised on the human or animal body, said method comprising the steps of
i) determining the blood coagulation activity of an individual according to the method of claim 13,
ii) correlating said blood coagulation activity of said individual determined in step i) said clinical condition, and
iii) based on the correlation of step ii), diagnosing said clinical condition in said human or animal body.

85. Method of claim 81, wherein said clinical condition in said individual is an acute clinical condition.

86. Method of claim 81, wherein said clinical condition in said individual is a condition developing into an acute clinical condition unless treated.

87. Method of claim 81, wherein said clinical condition in said individual is selected from the group consisting of cardiovascular diseases.

88. Method of claim 87, wherein said cardiovascular disease is selected from venous thromboembolism, pulmonary embolism, myocardial infarction, angina pectoris, coronary heart disease, disseminated intravascular coagulation,

89. Method of claim 81, wherein said clinical condition in said individual is selected from the group consisting of a liver disease, a malignant disease, acute pro-myelocytic leukaemia, diabetes, respiratory distress syndrome, and cerebral infarction.

90. System for and quantification of at least one blood coagulation activity marker present in a body fluid sample, and information linking said determined amount of said blood coagulation activity marker to the blood coagulation activity of an individual, comprising
i) a zone for applying a body fluid sample comprising a blood coagulation activity marker, said zone comprising at least one movable reporter species capable of binding said marker, said application zone being in liquid contact with
ii) a zone for detecting the presence, amount or concentration of said at least one reporter species bound to said marker, said zone further comprising a binding species for immobilizing onto said detection zone at least a substantial amount of said marker comprised in said body fluid sample, and optionally
iii) a positive control zone generating a positive control confirming the transfer of at least part of said body fluid sample from said application zone to said detection zone.

91. System according to claim 90, wherein the at least one reporter species comprises at least one antibody.

92. System according to claim 90, comprising
i) a hollow casing having a body fluid sample application aperture and a test result observation aperture,
ii) a bibulous body fluid sample receiving member within said hollow casing to receive said body fluid sample applied to said sample application aperture,
iii) a test strip comprising a dry porous carrier such as nitrocellulose within said casing and extending from said bibulous body fluid sample receiving member to and beyond said test result observation aperture, said dry porous carrier having a test result zone observable through said observation aperture,
iv) at least one of said bibulous body fluid sample receiving member and said test strip containing upstream from said test result zone a detectable reporter species capable of specifically binding said marker to form a first complex,
v) said reporter species comprising ad least one particulate label, such as a dye sol, a metallic sol or a coloured latex particle, and optionally also at least one fluorescently detectable label, said label being released into a mobile form by said body fluid sample,
wherein mobility of said label within said test strip is facilitated by either coating at least a portion of said test strip upstream from said test result zone with a material comprising a polysaccharide, or drying said label onto a portion of said test strip upstream frog said test zone in the presence of a material comprising a polysaccharide, in an amount effective to reduce interaction between said test strip and said label, and
wherein said dry porous carrier contains in said test result zone a means for binding said first complex, said means for binding comprising specific binding means immobilized in said test result zone, and
wherein migration of said body fluid sample from said bibulous sample receiving member into and through said dry porous carrier conveying by capillarity said first complex to said test result zone of said dry porous carrier whereat said binding means binds said first complex thereby to form a second complex, and
vi) determining the presence, amount or concentration of said second complex being observable through said test result observation aperture.

93. System according to claim 90, wherein such determination is based on a cut-off point, above which one visible colour indicates the presence of the marker and below which cutoff point another visible colour indicates that the marker is present in an amount less than that indicated cut-off point.

94. System of claim 93, wherein said cut-off point is at least 0.1 nM, for example at least 0.15 nM, such as at least 0.20 nM, for example at least 0.25 nM, such as at least 0.30 nM, for example between 0.1 and 2.0 nM, for example between 0.20 and 1.5 nM, such as between 0.30 and 1.0 nM.

95. System of claim 93, wherein said cut-off point is around 0.30 nM.

96. System of claim 90, wherein said biological species is selected from the group consisting of pro-thrombin, thrombin, thrombin antithrombin II complex (TAT), fibrinogen, fibrin, fibrin/fibrinogen degradation products D-dimer (FDP D-dimer), and alpha 2PI plasmin complex (PIC).

97. System of claim 96, wherein said blood coagulation activity marker is selected from the group consisting of peptides comprising a fragment of pro-thrombin.

98. System of claim 97, wherein said marker is selected from the group consisting of peptides comprising prothrombin Fragment 1+2 (F₁+₂), peptides comprising pro-thrombin Fragment 1 (F₁), and peptides comprising pro-thrombin Fragment 2 (F₂).

99. System of claim 97, wherein said marker is selected from peptides comprising pro-thrombin Fragment 1+2 (F₁)

100. System of claim 97, wherein said marker is selected from peptides comprising pro-thrombin Fragment 1 (F₂).

101. System of claim 97, wherein said marker is selected from peptides comprising pro-thrombin Fragment 1 (F₂).

102. System of claim 24, wherein said marker essentially consists of pro thrombin Fragment 1+2 (F1).

103. System of claim 90, wherein said marker is detectable by a reporter species capable of detecting any of pro-thrombin Fragment 1+2 (F₁₊₂), pro-thrombin Fragment 1 (F₁), and pro-thrombin Fragment 2 (F₂).

104. System of claim 90, wherein said blood coagulation activity marker is selected from the group consisting of peptides comprising a fragment of fibrinogen.

105. System of claim 95, wherein said marker is selected from the group consisting of peptides comprising fibrinopeptide A (FpA).

106. System of claim 95, wherein said marker essentially consists of fibrinopeptide A (FpA).

107. System of claim 95,1 wherein said marker is fibrinopeptide A (FpA).

108. System of claim 95, wherein said marker is detectable by a reporter species capable of detecting fibrinopeptide A (FpA).

109. System of claim 90, wherein said marker is selected from the group consisting of peptides comprising the carboxy-terminal 17 amino acid residues of the heavy chain of Factor Xₐ.

110. System of claim 90, wherein said reporter species comprises at least one targeting species,

111. System according to claim 90, wherein said targeting species comprises at least one antibody, or a binding fragment thereof, capable of detecting at least one blood coagulation marker defined by an antibody against F₁₊₂.

112. System of claim 111, wherein said targeting species comprises the F₁₊₂ antibody deposited with the ATCC under the Accession No. HB 10291, or a binding fragment thereof, capable of detecting said blood coagulation marker.

113. System according to 90, wherein said targeting species comprises at least one antibody, or a binding fragment thereof, capable of detecting at least one blood coagulation marker defined by an antibody against F₁.

114. System of claim 113, wherein said targeting species comprises the antibody against F₁ disclosed by Bezeaud and Guillin in British Journal of Haematology (1984), vol. 58, pages 597-606, on, a binding fragment thereof, capable of detecting said blood coagulation marker.

115. System according to 90, wherein said reporter species comprises at least one antibody, or a binding fragment thereof, capable of detecting at least one blood coagulation marker defined by an antibody against F₂.

116. System of claim 115, wherein said targeting species comprises the antibody against F₂ deposited with the ATCC under the Accession No. HB 10291, or a binding fragment thereof, capable of detecting said blood coagulation marker.

117. System according to 90, wherein said targeting species comprises at least one antibody capable of detecting at least one blood coagulation marker defined by an antibody against FpA.

118. System of claim 117, wherein said targeting species comprises a binding fragment of said FpA antibody capable of detecting said blood coagulation marker.

119. System according to claim 90, wherein said targeting species comprises at least one antibody capable of detecting at least one blood coagulation marker defined by an antibody against Xₐ.

120. System of claim 119, wherein said targeting species comprises a binding fragment of said Xₐ antibody capable of detecting said blood coagulation marker.

121. System according to any of the claims 90, wherein the targeting species is immobilised on a solid surface.

122. System according to claim 121, wherein said solid surface is comprised within a lateral flow device.

123. System according to claim 121, wherein said solid surface is a dipstick or part thereof.

124. System according to claim 121, wherein said solid surface is nitrocellulose.

125. System according to claim 121, wherein said solid surface is comprised within a micro fluid device,

126. System according to claim 90, wherein said reporter species further comprises at least one polypeptide operably linked to said at least one targeting species.

127. System of claim 126, wherein said polypeptide comprises an enzyme.

128. System of claim 127, where said enzyme comprises a peroxidase activity.

129. System according to claim !90, wherein said reporter species further comprises at least one fluorochrome.

130. System according to claim 90 wherein said reporter species further comprises at least one radio label.

131. System according claim 90, wherein said reporter species further comprises at least one coloured dye molecule.

132. System according to claim 90, wherein said reporter species comprises two antibodies.

133. System according to claim 90, wherein said two antibodies are selected from a polyclonal antibody and a monoclonal antibody.

134. System according to claim 90, wherein said reporter species comprises a polymeric carrier molecule.

135. System according to 134, wherein the polymeric carrier molecule has a hydrophilic sugar chain backbone.

136. System according to 134, wherein the polymeric carrier molecule has a polymeric dextran backbone.
